(19) United States
(12) Patent Application Publication
(10) Pub. No.: US 2001/0031859 A1
(43) Pub. Date: Oct. 18, 2001

(54) High level cytokine production with enhanced cell viability

(76) Inventors: Allan S. Lau, Fremont, CA (US); Natalya Ossina, Albany, CA (US); Kenneth Hoyt, San Rafael, CA (US)

Correspondence Address: IOTA PI LAW GROUP, 350 CAMBRIDGE AVENUE SUITE 250, P O BOX 60850, PALO ALTO, CA, 94306-0850, US

(21) Appl. No.: 09/772,109

(22) Filed: Jan. 26, 2001

Related U.S. Application Data
(63) Continuation-in-part of application No. 09/657,881, filed on Sep. 8, 2000.
(60) Provisional application No. 60/152,854, filed on Sep. 8, 1999.

(51) Int. Cl. C12P 21/02; C12N 5/08; C07K 14/52

(52) U.S. Cl. 530/351; 435/69.5; 435/366

(57) ABSTRACT

The present invention relates to compositions and methods for enhanced cytokine production in human cell culture, particularly under conditions where apoptotic cell death is suppressed by expression of CrmA.

This application is a continuation-in-part of U.S. application Ser. No. 09/657,881 filed on Sep. 8, 2000, which claims priority to U.S. Provisional Application Ser. No. 60/152,854 filed Sep. 8, 1999, both of which are hereby incorporated by reference in their entirety.

### FIELD OF THE INVENTION

The present invention relates to compositions and methods for enhanced cytokine production in human cell culture by inhibiting apoptosis associated with cytokine synthesis, particularly under conditions of cytokine regulatory factor overexpression.

### REFERENCES

Abbas, A K, et al., Eds., CELLULAR AND MOLECULAR IMMUNOLOGY, 3^{rd} edition, WB Saunders Co., 256-257, 1997.

Antonelli, G., J. Interferon Cytokine Res (17)Suppl 1:S39-S46, 1997.

Antonelli, et al., J. Inf. Disease 163:882-885 1991.

Ausubel, FM, et al., in CURRENT PROTOCOLS IN MOLECULAR BIOLOGY, John Wiley and Sons, Inc., Media, Pa. 1992.

Baker, S. J., et al., Science 249(4971):912-5, 1990.

Balachandran, S., et al., EMBO J. 17:6888-6902, 1998.

Balkwill F R and Burke F, Immunology Today, 10(9):299, 1989.

Boise, Thompson. Current Topics Microbiol Immunol 200:107-121, 1995.

Brenner B, et al., Cell Death Differ 5(1):29-37, 1998.

Chinnaiyen, D., et al., Cell 81:505-512, 1995.

Chinnaiyan, A. M et al., J. Biol. Chem. 271, 4573-4576, 1996.

Chinnaiyan, A. M. et al., J. Biol. Chem. 271:4961-4965, 1996.

Chong K L et al., EMBO J, 11(4):1553-62 1992.

Clemens M J and Bommer U A, Int J Biochem Cell Biol, 31(l):1-23, 1999.

Clemens M J and Elia A, J Interferon Cytokine Res, 17(9):503-24, (1997).

Clark S and Kamen R, Science, 236:1229-1237, 1987.

Cohen, J. J., Immunol Today 14(3):126-130, 1993.

Cohen, J. J., et al., Annu Rev Immunol 10:267-293, 1992.

Dbaibo G S et al., J. Exp. Med., 185(3):481-90, 1997.

Dbaibo G S and Hannun Y A. Clin Immunol Immunopathol. 86(2):134-40, 1998.

Der, D., and Lau, A. S., Proc Natl Acad Sci USA, 92:8841-8845, (1995).

Deutscher, METHODS IN ENZYMOLOGY, 182, 1990; Scopes, PROTEIN PURIFICATION: PRINCIPLES AND PRACTICE, Springer-Verlag, New York, 1982.

Donze, O., et al., Virol 256:322-329, 1999.

Dressler, K. A., et al., Science 255:1715-1718, 1992.

Feng G S et al., Proc Natl Acad Sci USA 89(12):5447-51, 1992.

Galabru, J., and Hovanessian, A., J. Biol. Chem. 262:15538-15544, 1987.

Garcia, I., et al., Science 258:302-304, 1992.

Guy, G. R., et al., J Biol Chem 267(3):1846-1852, 1992.

Haimovitz-Friedman, A, et al., J. Exp. Med. 180:525-535, 1994.

Harlow and Lane, ANTIBODIES: A LABORATORY MANUAL, Cold Spring Harbor Pubs., N.Y. 1988.

Heller, R. A., et al., Cell 70(1):47-56, 1992.

Hershey, J. W. B., Ann. Rev. Biochem. 60:717-755, 1991.

Hopp et al., Biotechnology 6:1204-1210, 1988.

Huang et al., Oncogene 14: 405-414,1997.

Inoue I et al. Neurol Med Chir (Tokyo) 31(8):485-9, 1991.

Jagus R. et al., Int J Biochem Cell Biol. 31(1): 123-38, 1999.

Jayadev, S, et al., J. Biol. Chem. 270: 2047-2052, 1995.

Kane, D. J., et al., Science 262:1274-1277, 1993.

Korsmeyer, S. J., Blood 80(4):879-886, 1992.

Koromilas et al., Science 257:1685, 1992.

Kosik, K. S., Science 256:780-783, 1992.

Kostura, MJ et al., Proc. Natl. Acad. Sci. U.S.A. 86, 5227-5231, 1989.

Kumar, A., et al Proc Natl Acad Sci USA 91:6288-6292, 1994.

Larrick, J. W., and Wright, S. C., FASEB J4:3215-3223, 1990.

Lau, A. S., et al., Pediat Infect Dis J, CME Review 15:563-575, 1996.

Levine, A. J., Annu Rev Biochem 62:623-651, 1993.

Liddil, J. D., et al., Cancer Res 49:2722-2728, 1989.

Meurs EF et al., J Virol. 66(10):5804-14 1992.

Meurs, E., and Hovanessian, A. G., Cell 62:379-390, 1990.

Miura, M et al., Cell. 75: 653-660, 1993.

Mulligan and Berg, 1980.

Muzio, M et al., Cell 85, 817-827, 1996.

Nagata, S., and Suda, T., Immunol Today 16(1):39-43, 1995.

Nicholson, D. W et al., Nature 376, 37-43, 1995.

Obeid, L. M., et al., Science 259:1769-1771, 1993.

Oehm, A., et al., J Biol Chem 267(15):10709-10715, 1992.

Orrenius, S., J Intern Med 237:529-536, 1995.

Orth, K et al., J. Biol. Chem. 271, 16443-16446, 1996.

Reed, J., et al., Nature 336:259-261, 1988.

Reed, J., et al., Exp Cell Research 195:277-283, 1991.

Rubin, B. Y., et al., Cancer Res 48:6006-6010, 1988.

Sambrook J, et al., in MOLECULAR CLONING: A LABORATORY MANUAL, Cold Spring Harbor Laboratory Press, Cold Spring Harbor, N.Y., 1989.

Schendel, Cell Death Differentiation 5(5):372-380, 1998.

Schulze-Osthoff, K., et al., Eur J Biochem 254:439-459, 1998.

Sen, G. C., and Lengyel, P., J Biol Chem 267:5017-5020, 1992.

Southern and Berg, J Mol Appl Genet. 1(4):327-41, 1982.

Stellar, H., Science 267:1445-1449, 1995.

Srivastave, S., et al., J Biol Chem 273:2416-2423, 1998.

Sugden et al., 1985.

Tan S L, Gale M J Jr, Katze M G, Mol Cell Biol 18(5):2431-43, 1998.

Taylor, J. L., and Grossberg, S. E., Virus Research 15:126, 1990.

Tepper, C. G, et al., Proc. Natl. Acad. Sci. USA. 92: 8443-8447,1995.

Tewari, M et al., Cell 81, 801-809,1995.

Tewari, M et al., J. Biol. Chem. 270: 3255-3260, 1995.

Thompson, C. B., Science 267:1456-1462, 1995.

Tracey, K. J., and Cerami, A., Annu Rev Cell Biol. 9:317-343, 1993.

Van Lint, J., et al., J Biol Chem 267(36):25916-25921, 1992.

Vaux, D. L., Proc Natl Acad Sci USA 90:786-789, 1993

Wegenknecht B et al., Cell Death Differ 5:894-900, 1998.

Wek R C, Trends Biochem Sci; 19: 491-496, 1994.

Williams, B. R. G., Eur J Biochem. 200:111, 1991.

Williams, B. R. G., Seminars in Oncology 24(S9):70-77, 1997.

Williams B R, Oncogene, 18(45):61 12-20, 1999.

Wong G and Clark S, Immunology Today, 2(5):137, 1988.

Yeh, et al., Science 279:1954-1958, 1998.

Yeung, M. C., et al., Proc Natl Acad Sci USA 93:12451-12455, 1996.

Yeung, M., and Lau, A. S., J Biol Chem 273:25198-25202, 1998.

Yeung, M., et al., AIDS 12:349-354, 1998.

Yonehara, S., et al., J Exp Med 169(5):1747-1756, 1989.

Zamanian-Daryoush M, et al., Oncogene 14;18(2):315-26, 1999.

Zamanian-Daryoush, et al., Molecular and Cellular Biology, 20:1278-1290, 2000.

Zhou Q, et al., J Biol Chem 272(12):7797-800, 1997.

### BACKGROUND OF THE INVENTION

Infection by pathogens including viruses, bacteria, and parasites results in activation of the host immune system and signaling by various molecules, such as cytokines, resulting in mobilization of multiple branches of the immune system. Cytokines are a rapidly growing collection of potent, pleiotropic polypeptides that act as local and/or systemic intercellular regulatory factors. (See, e.g., Balkwill and Burke, 1989; Wong and Clark, 1988; and Clark and Kamen, 1987.) They play crucial roles in many biologic processes, such as immunity, inflammation, and hematopoiesis, and are produced by diverse cell types including fibroblasts, endothelial cells, macrophages/monocytes, and lymphocytes. To date, a large number of cytokines have been identified, including interferons (IFNs), tumor necrosis factors (TNFs), interleukins (ILs), growth factors such as epidermal growth factors and differentiating factors, such as colony stimulating factors (CSF).

In general, cytokines and numerous other proteins which have both pharmaceutical and industrial application are produced by either purifying the natural protein from cell culture or recombinantly producing the protein in insect, microbial or human cells. Natural cytokines and other proteins are preferable in that they are known to contain the full repertoire of native forms of a given cytokine or protein and have the proper structure. However, such native form cytokines and proteins are expensive and time-consuming to produce.

Recombinantly produced cytokines and other proteins are less expensive to make, but may contain foreign antigens, resulting in an immune response by the subject to which they are administered, or may be less active due to structural variation relative to the native form, i.e., glycosylation pattern. In general, present methods for production of cytokines and other proteins are based on expression of these factors: (i) in microbial systems, which may not permit the proper glycosylation for native folding of the proteins, or (ii) in human cells with low production levels.

Thus, a method for enhancing the production of natural cytokines and other proteins which makes them less expensive to produce would be advantageous.

dsRNA-activated protein kinase (PKR) referred to as P1/e1F2 kinase, DAI or dsI for dsRNA-activated inhibitor, and p68 (human) or p65 (murine) kinase, is a serine/threonine kinase whose enzymatic activation requires binding to dsRNA or to single-stranded RNA presenting internal dsRNA structures and consequent autophosphorylation (Galabru and Hovanessian, 1987; Meurs, et al., 1990). PKR plays a key role in the expression of a number of useful cytokines including interferons, as described in U.S. Pat. No 6,159,712, expressly incorporated by reference herein.

It has also been suggested that PKR may function as a tumor suppressor and inducer of apoptosis. (See, e.g., Clemens and Bommer, 1999; Koromilas, et al., 1992), with recent results indicating that expression of an active form of PKR triggers apoptosis, possibly through upregulation of the Fas receptor (Donze, O., et al., 1999). See, also Yeung, M. C., et al., 1996; Yeung, M., and Lau, A. S., 1998).

Taken together, these results suggest that it would be desirable to inhibit apoptotic cell death in PKR-expressing cell lines, in order to prolong the lifespan of the cells during cytokine induction and thereby enhance the production of cytokines and other proteins by the cells.

### SUMMARY OF THE INVENTION

The invention includes, in one aspect, a method for producing one or more cytokines in mammalian cell culture.

The invention provides human cell compositions for the production of cytokines, where the cells are characterized by expression of the coding sequence for an anti-apoptotic protein and a level of cytokine production that is at least two times (2×) the level of cytokine production exhibited by the corresponding parental cell line that does not express the anti-apoptotic protein.

In a related aspect the invention provides methods for producing human cell compositions that exhibit enhanced activity, expression or production of one or more cytokines selected from the group consisting of interferon-alpha (IFN-alpha), interferon-beta (IFN-beta), interferon-gamma (IFN-gamma); granulocyte macrophage colony stimulating factor (GM-CSF); granulocyte colony stimulating factor (G-CSF); interleukin-2 (IL-2); interleukin-3 (IL-3); interleukin-7 (IL-7); interleukin-8 (IL-8); interleukin-10 (IL-10); and interleukin-12 (IL-12).

Preferred anti-apoptotic proteins for expression in such cytokine-producing cells include modified or mutant forms of eIF-2a, FADD, Bcl-X_{S}, BAK or BAX; anti-apoptotic proteins such as Bcl-2, Bcl-X_{L} and related homologues, in particular, CrmA.

Such cytokine-producing human cell compositions may be prepared by modifying cells of a parental human cell line capable of producing cytokines by introducing a first expression vector comprising the coding sequence for CrmA and a selectable marker-encoding nucleic acid sequence and culturing the modified cells in the presence of a selection agent to select for CrmA-expressing cells.

In practicing the invention, anti-apoptotic protein-producing cells may be further (1) treated in a manner effective to result in enhanced cytokine production, by priming and/or induction; or (2) modified by introducing a second expression vector comprising the coding sequence for a cytokine regulatory factor such as PKR and a selectable marker-encoding nucleic acid sequence, selection for CrmA- and PKR-expressing cells, followed by priming and/or induction.

Priming may be accomplished by exposing the transformed cells to any of a number of agents, such as phorbol myristate acetate (PMA) or interferon-β. Induction means exposing the transformed cells to a microbial inducing agent, such as Sendai virus, encephalomyocarditis virus or Herpes simplex virus; or exposing the cells to at least one non-microbial inducing agent selected from the group consisting of poly(I):poly(C) (poly IC), or poly r(I):poly r(C) (poly rIC), heparin, dextran sulfate, cycloheximide, Actinomycin D, sodium butyrate, calcium ionophores and chondroitin sulfate.

By inhibiting apoptosis, the cell line compositions and methods of the invention exhibit an increase in cytokine production and/or an increase in the time over which the cells function to produce cytokines.

These and other objects and features of the invention will become more fully apparent when the following detailed description of the invention is read in conjunction with the accompanying drawings.

### BRIEF DESCRIPTION OF THE FIGURES

FIG. 1 shows the results of a propidium iodide assay for cell viability in parental wild type (WT) and CrmA-expressing (CrmA-#2) MG-63 cells following superinduction and viral induction with Sendai virus.

FIG. 2 shows interferon-beta production in parental wild type (WT) and CrmA-expressing (CrmA-#2) MG-63 cells following superinduction and Sendai virus treatment.

FIG. 3 shows the effect of 0, 2mM, 4mM, and 8 mM 2-aminopurine (2-AP; a PKR inhibitor) on interferon-beta production in CrmA-expressing (CrmA-#2) MG-63 cells following superinduction.

FIGS. 4A and 4B show the percentage of viable 6A, A9 and WT cell lines following cytokine induction by Sendai virus and poly IC, respectively.

FIGS. 5A and 5B show the IFN-alpha levels produced in 6A, A9 and WT cell lines following treatment with Sendai virus and poly IC, respectively.

### DETAILED DESCRIPTION OF THE INVENTION

I. Definitions

Unless otherwise indicated, all technical and scientific terms used herein have the same meaning as they would to one skilled in the art of the present invention. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

All publications cited herein are expressly incorporated herein by reference for the purpose of describing and disclosing compositions and methodologies which might be used in connection with the invention.

A “heterologous” nucleic acid construct or sequence has a portion of the sequence which is not native to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous” nucleic acid construct may contain a control sequence/DNA coding sequence combination that is the same as, or different from a control sequence/DNA coding sequence combination found in the native cell.

The terms “vector”, as used herein, refer to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art. A cloning or expression vector may comprise additional elements, e.g., the expression vector may have two replication systems, thus allowing it to be maintained in two organisms, e.g. in human cells for expression and in a prokaryotic host for cloning and amplification. Cloning and expression vectors will typically contain a selectable marker.

As used herein, the term “selectable marker-encoding nucleotide sequence” refers to a nucleotide sequence which is capable of expression in mammalian cells and where expression of the selectable marker confers to cells containing the expressed gene the ability to grow in the presence of a selective agent.

As used herein, the term “promoter” refers to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences. A promoter may be constitutive or inducible and may be a naturally occurring, engineered or hybrid promoter.

The term “control sequences” refers to DNA sequences necessary for the expression of an operably linked coding sequence in a particular host organism. The control sequences that are suitable for prokaryotes, e.g., include a promoter, optionally an operator sequence, and a ribosome binding site. Eukaryotic cells are known to utilize promoters, polyadenylation signals, and enhancers.

As used herein, the term “operably linked” relative to a recombinant DNA construct or vector means nucleotide components of the recombinant DNA construct or vector that are directly linked to one another for operative control of a selected coding sequence. Generally, “operably linked” DNA sequences are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “gene” means the segment of DNA involved in producing a polypeptide chain, which may or may not include regions preceding and following the coding region, e.g. 5′ untranslated (5′ UTR) or “leader” sequences and 340 UTR or “trailer” sequences, as well as intervening sequences (introns) between individual coding segments (exons).

As used herein, the term “sequence identity” means nucleic acid or amino acid sequence identity in two or more aligned sequences, aligned using a sequence alignment program.

The term “% homology” is used interchangeably herein with the term “% identity” herein and refers to the level of nucleic acid or amino acid sequence identity between two or more aligned sequences, when aligned using a sequence alignment program. E.g., as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90 or 95% or more sequence identity to a PKR sequence, as described herein.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at http://www.ncbi.nlm.nih.gov/BLAST/”. See, also, Altschul, S. F. et al., 1990 and Altschul, S. F. et al., 1997.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences which have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. [See, Altschul, et al., 1997.]

A preferred alignment of selected sequences in order to determine “% identity” between two or more sequences, is performed using e.g., the CLUSTAL-W program in MacVector version 6.5, operated with default parameters, including an open gap penalty of 10.0, an extended gap penalty of 0.1, and a BLOSUM 30 similarity matrix.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. E.g., “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

Moderate and high stringency hybridization conditions are well known in the art (see, e.g., Sambrook, et al., 1989, Chapters 9 and 11, and in Ausubel, F. M., et al., 1993, expressly incorporated by reference herein). An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C.

As used herein, “recombinant” includes reference to a cell or vector, that has been modified by the introduction of a heterologous nucleic acid sequence or that the cell is derived from a cell so modified. Thus, e.g., recombinant cells express genes that are not found in identical form within the native (non-recombinant) form of the cell or express native genes that are otherwise abnormally expressed, under expressed or not expressed at all as a result of deliberate human intervention.

As used herein, the terms “transformed”, “stably transformed” or “transgenic” with reference to a mammalian cell means the mammalian cell has a non-native (heterologous) nucleic acid sequence integrated into its genome which is maintained through two or more generations.

As used herein, the term “expression” refers to the process by which a polypeptide is produced based on the nucleic acid sequence of a gene. The process includes both transcription and translation.

The term “cytokine regulatory factor expression” refers to transcription and translation of a cytokine regulatory factor gene, the products of which include precursor RNA, mRNA, polypeptide, post-translation processed polypeptide, and derivatives thereof, and including cytokine regulatory factors from other species such as murine or simian enzymes.

It follows that the term “PKR expression” refers to transcription and translation of a PKR encoding nucleic acid sequence, the products of which include precursor RNA, mRNA, polypeptide, post-translation processed polypeptide, and derivatives thereof, and including PKRs from other species such as murine or simian enzymes. By way of example, analyses for PKR expression include autophosphorylation assays and eIF2α phosphorylation assays for PKR activity, Western blot for protein expression, and Northern blot analysis and reverse transcriptase polymerase chain reaction (RT-PCR) for PKR mRNA expression.

“Alternative splicing” is a process whereby multiple polypeptide isoforms are generated from a single gene, and involves the splicing together of nonconsecutive exons during the processing of some, but not all, transcripts of the gene. Thus a particular exon may be connected to any one of several alternative exons to form messenger RNAs. The alternatively-spliced mRNAs produce polypeptides (“splice variants”) in which some parts are common while other parts are different.

As used herein, the terms “biological activity” and “biologically active”, refer to the activity attributed to a particular protein in a cell line in culture. It will be appreciated that the “biological activity” of such a protein may vary somewhat dependent upon culture conditions and is generally reported as a range of activity. Accordingly, a “biologically inactive” form of a protein refers to a form of the protein which has been modified in a manner which interferes with the activity of the protein as it is found in nature.

As used herein, the terms “biological activity of a cytokine regulatory factor” and “biologically active cytokine regulatory factor” refer to any biological activity associated with the a particular cytokine regulatory factor or any fragment, derivative, or analog of that cytokine regulatory factor such as enzymatic activity, etc.

As used herein, the terms “normal level of cytokine regulatory factor activity” and “normal level of cytokine regulatory factor expression” refer to the level of cytokine regulatory factor activity or expression, determined to be present in unmodified, uninduced, unprimed or uninfected cells of a particular type, e.g., the parental cell line of a particular type. It will be appreciated that such “normal” cytokine regulatory factor activity or expression, is reported as a range of cytokine regulatory factor activity or expression which is generally observed for a given type of cells that have not been transfected with a vector encoding cytokine regulatory factor, are unstimulated (not induced or primed) and uninfected.

It follows that the terms “biological activity of PKR” and “biologically active PKR” refer to any biological activity associated with PKR, or a fragment, derivative, or analog of PKR, such as enzymatic activity, specifically including autophosphorylation activity and kinase activity involving phosphorylation of substrates such as eukaryotic translation initiation factor 2 (eIF-2) and transcription factors such as NF-κB.

Similarly, the terms “normal level of PKR activity” and “normal level of PKR expression” refer to the level of PKR activity or expression, determined to be present in unstimulated or uninfected cells of a particular type, e.g., a particular cell line. It will be appreciated that such “normal” PKR activity or expression, is reported as a range of PKR activity or expression which is generally observed for a given type of cells that have not been transfected with a vector encoding PKR, are unstimulated (not induced or primed) and uninfected.

The range of “normal” cytokine regulatory factor activity or expression may vary somewhat dependent upon culture conditions. For example, the U937 cell line may have a normal range of PKR activity which differs from the normal range of PKR activity for the Vero or Namalwa cell lines. It follows that over-expression of PKR means an expression level which is above the normal range of PKR expression generally observed for a given type of cells which are not transfected with a vector encoding PKR, unstimulated (not induced or primed) and uninfected. Accordingly, “overexpression” of PKR means a range of PKR activity, expression or production which is greater than that generally observed for a given type of cells which have not been modified by introduction of a vector comprising the coding sequence for PKR, selected for PKR overexpression, are unstimulated (not induced or primed) and are uninfected.

In one preferred aspect, cytokine regulatory factor overexpression means a level of cytokine regulatory factor activity, expression or production that is at least 150% (1.5-fold or 1.5×), preferably at least 200%, 300% or 400%, or 500% or more greater than the normal level of cytokine regulatory factor activity, expression or production for the same cell line under the particular culture conditions employed. In other words, a cell line that over expresses a cytokine regulatory factor typically exhibits a level of cytokine regulatory factor production or expression that is at least 1.5-fold and preferably 2-fold (2×), 3-fold (3×), 4-fold (4×), 5-fold (5×) or more greater than the level of cytokine regulatory factor expression or production typically exhibited by the same type of cells which have not been selected, modified or treated in a manner effective result in cytokine regulatory factor overexpression.

In some cases, a cell line that over expresses a cytokine regulatory factor such as PKR exhibits a level of cytokine regulatory factor expression or production that is 10-fold (10×) or more greater than the level of cytokine regulatory factor expression or production typically exhibited by the same type of cells under the particular culture conditions employed and which have not been selected or treated in a manner effective result in cytokine regulatory factor overexpression. By way of example, the term “treated in a manner effective result in PKR overexpression”, means one or more of introduction of a PKR coding sequence (of heterologous or autologous origin) into the cell, selection, stimulation (priming or priming and induction) and/or infection.

As used herein, the terms “normal level of cytokine” and “normal level of protein”, relative to activity, expression, and production, refer to the level of cytokine or other protein activity, expression or production, determined to be present in cells of a particular type which have not been treated in a manner effective result in cytokine regulatory factor overexpression. Examples include, a wild type cell line which has not been selected or treated in a manner to result in enhanced cytokine regulatory factor activity, expression or production and a cell line which does not comprise an introduced cytokine regulatory factor coding sequence. It will be appreciated that such “normal” cytokine or other protein activity, expression, or production, is reported as a range of activity, expression, or production, typically observed for a given type of cells and may vary somewhat dependent upon culture conditions.

Accordingly, the range of “normal” cytokine activity or expression may vary somewhat dependent upon culture conditions. The terms an “enhanced level of” and “above normal level of” relative to cytokine or protein activity, expression or production may be used interchangeably. The terms refer to a level of cytokine or protein activity, expression or production that is at least 150% (1.5-fold or 1.5×), preferably at least 200%, 300% or 400%, or 500% or more greater than the level of cytokine or protein activity, expression or production exhibited by parental cells of the same cell line under the particular culture conditions employed, where the parental cells have not been selected, modified or treated in a manner effective result in an increase in cytokine or protein activity, expression, or production. In some cases, the increase in cytokine or protein activity, expression, or production is 10-fold (10×) or more greater than that of the parental cell line.

As used herein the terms “purified” and “isolated” generally refer to molecules, either polynucleotides or polypeptides, that are separated from other components of the environment in which they were found or produced. For example an isolated or purified polynucleotide or polypeptide has typically been separated from 75% or more of the components of the environment in which they were found or produced. An isolated or purified polynucleotide or polypeptide has preferably been separated from at least 80% to 85% and more preferably at least 90%, 95% or more of the components of the environment in which they were found. For example, a “purified” or “isolated” cytokine means the cytokine has been separated from at least 75% or more, preferably from at least 80% to 85% or more and more preferably from at least 90% or more of the components in the cell culture medium in which they were produced.

The terms “apoptotic cell death”, “programmed cell death” and “apoptosis”, as used herein refer to any cell death that results from, or is related to, the complex cascade of cellular events that occur at specific stages of cellular differentiation and in response to specific stimuli. Apoptotic cell death is characterized by condensation of the cytoplasm and chromatin condensation in the nucleus of dying cells. The process is associated with fragmentation of DNA into multiples of 200 base pairs and degradation of RNA as well as proteolysis in an organized manner without sudden lysis of the cell as in necrotic cell death.

As used herein, the term “inhibit apoptotic cell death”, means to partially or completely inhibit the cell death process over the time period a cell line is cultured for the purpose of cytokine or other protein expression or production. Such inhibition generally means the amount of apoptotic cell death is decreased by at least 20%, preferably by at least 50% and more preferably by 80% or more relative to the amount of apoptotic cell death observed in a cell line which has not been modified in a manner effective to inhibit apoptosis.

In the case of cytokine-producing cell lines, such inhibition generally means the amount of apoptotic cell death is decreased by at least 20%, preferably by at least 50% and more preferably by 80% or more relative to the amount of apoptotic cell death observed in a PKR-overexpressing cell line which has not been modified in a manner effective to inhibit apoptosis.

The definitions set forth above with respect to cytokines also apply to “other proteins”, produced by the methods of the invention, such as CrmA, Bcl-2, Bcl-X_{L} and related homologues. Accordingly, “overexpression” of CrmA, Bcl-2 or Bcl-X_{L}, respectively, means a range of CrmA, Bcl-2 or Bcl-X_{L} activity or expression which is greater than that generally observed for a given type of cells which have not been transfected with a vector encoding CrmA, Bcl-2 or Bcl-X_{L}, and stimulated to undergo apoptosis.

As used herein, the term “modified form of”, relative to proteins associated with apoptosis, exemplified by, eIF-2a or eIF-2alpha, eIF-3, FADD, Bcl-X_{S}, BAK, BAX, etc., means a derivative or variant form of the native protein. That is, a “modified form of” a protein has a derivative polypeptide sequence containing at least one amino acid substitution, deletion or insertion, with amino acid substitutions being particularly preferred. The amino acid substitution, insertion or deletion may occur at any residue within the polypeptide sequence, which interferes with the biological activity of the protein. The corresponding nucleic acid sequence which encodes the variant or derivative protein is considered to be a “mutated” or “modified form of” the gene or coding sequence therefor, and is included within the scope of the invention.

II. Cytokine Regulatory Factors and Cytokine Production

A number of factors are known to be involved in the induction and/or enhanced expression of cytokines in cells, e.g., human cells. These factors include cytokine- and other protein-specific transcriptional regulatory factors, e.g. interferon regulatory factors (IRF-1, IRF-3 and IRF-7), cytokine receptors, nuclear factor KB (NF-κB), activator protein-1 (AP-1), nuclear factor IL-6 (NF-IL6), and in particular, PKR.

Enhancing the expression or activity of any of these factors will generally result in higher than normal expression of one or more cytokine-encoding genes.

PKR is used as herein as an example of a protein capable of regulating cytokine and other protein expression; however, it will be understood that other cytokine and protein enhancing factors (designated herein as “cytokine regulatory factors” or “CRF”) may be used in place of PKR, e.g., (1) protein kinase C (PKC) inducers, TNF-α, GM-CSF, EGF and PDGF, G-CSF, TGF, TNF-alpha or TNF-beta, IL-1, IFNs (IFN-alpha, IFN-beta, IFN-gamma) or chemokines (IL-8, Macrophage inflammatory proteins [MIP-1a & -1b] and monocyte chemotactic proteins [MCPs]); (2) other cellular signaling factors such as PMA, calcium ionophores, sodium butyrate or endotoxin; (3) polyl: C, double-stranded RNA or viral analogs; (4) cellular stress signals that can activate PKR, including heat shock, pathogen infection, e.g. viral infection; or (5) any factor which enhances expression of such a cytokine regulatory factor resulting in enhanced cytokine production.

A. PKR

PKR is the only identified dsRNA-binding protein known to possess a kinase activity. PKR is a serine/threonine kinase whose enzymatic activation requires dsRNA binding and consequent autophosphorylation (Meurs, et al., 1990; Feng G S et al., 1992).

The best characterized in vivo substrate for PKR is the alpha subunit of eukaryotic initiation factor-2 (eIF-2a) which, once phosphorylated, ultimately leads to inhibition of cellular and viral protein synthesis (Hershey, J. W. B., 1991). PKR has been demonstrated to phosphorylate initiation factor e1 F-2 alpha in vitro when activated by double-stranded RNA (Chong, et al., 1992). This particular function of PKR has been suggested as one of the mechanisms responsible for mediating the antiviral and anti-proliferative activities of IFN-alpha and IFN-beta. An additional biological function for PKR is its putative role as a signal transducer, e.g., by phosphorylation of IkB, which results in the release and activation of nuclear factor kB (NF-kB) (Kumar A et al., 1994).

It has previously been demonstrated that PKR mediates the transcriptional activation of interferon (IFN) expression (Der D and Lau A S, 1995). IFNs elicit their biological activities by binding to their cognate receptors followed by signal transduction leading to induction of IFN-stimulated genes (ISGs). Such ISGs are believed to mediate the biological activities of IFNs by at least two intracellular pathways: degradation of RNA via the activation of a specific ribonuclease, and induction of an IFN-regulated, double stranded RNA-activated kinase (PKR). Consistent with this observation, suppression of endogenous PKR activity by transfecting U937 cells with an oligonucleotide antisense to PKR or expression of a PKR-deficient mutant resulted in diminished induction of IFN in response to viral infection (Der D and Lau A S, 1995).

In summary, PKR has been associated with (1) signal transduction for complex receptor systems (including IFN, TNF and Fas), (2) transcriptional activation of cytokine genes, (3) initiation of apoptosis, and (4) inhibition of protein synthesis by phosphorylating eIF-2α. Additional activities attributed to PKR include a role in (1) mediating the antiviral and anti-proliferative activities of IFN-alpha and IFN-beta, (2) the response of uninfected cells to physiologic stress, and (3) cell growth regulation (Clemens and Elia, 1997; Zamanian-Daryoush, et al., 1999).

It has also been suggested that PKR may function as a tumor suppressor and inducer of apoptosis. (See, e.g., Clemens M J et al., 1999; Yeung, Lau et al., 1996; Koromilas et al., 1992). Recent results indicate that expression of an active form of PKR triggers apoptosis, possibly through upregulation of the Fas receptor (Donze O, et al., 1999).

III. Apoptosis

Apoptosis or programmed cell death is a cell-intrinsic process that is a central part of normal development, is tightly regulated and important for development, host defense, and suppression of oncogenesis. (reviewed in Orrenius 1995; Stellar 1995; Vaux 1993). Apoptosis provides many advantages for organisms, both during fetal development (Cohen 1992), in controlling the formation of organs (Nagata et al., 1995; Vaux, 1993), and for purposes of homeostasis in adult life. Once committed to apoptosis, cells undergo new rounds of protein synthesis and various morphological/physiological changes including cytoplasmic condensation, nuclear chromatin condensation, membrane blebbing, and eventual DNA degradation at the internucleosomal linker sites yielding DNA fragments in multiples of 180 base pairs (bp), detected as a characteristic oligonucleosomal ladder (Levine A J, 1993). The dying cell eventually fragments into membrane-bound apoptotic bodies that are rapidly phagocytosed and digested by macrophages or by neighboring cells.

Apoptosis serves as a defense mechanism to remove unwanted and potentially dangerous cells including virus-infected cells, self-reactive lymphocytes in autoimmune diseases, or malignant cells (Oehm, et al., 1992; Yonehara, et al., 1989; Vaux, 1993). Apoptosis has been implicated as a means to minimize the risk of cancer cell development in tissues frequently exposed to mutagenic chemicals, carcinogens, or UV radiation.

A further protection against malignancy is afforded by TNF-α, a proinflammatory cytokine, produced in response to activation of the immune system. TNF-α can trigger the apoptotic death of transformed host cells (Heller, 1992, Yeung, 1996).

Deregulation of apoptosis may contribute to the pathogenesis of disease processes (Thompson, 1995). It is believed to play a critical role in disease development including cancer, AIDS, ischemic stroke and neurodegenerative disorders. Evidence suggests that inhibition of cell death and inappropriate cell death may both be deleterious to the host, for example in neurodegenerative diseases including Alzheimer and Parkinson diseases which are associated with the premature death of particular subtypes of neurons (Kosik K S, 1992). Inappropriate suppression or inherent deficiency of cellular apoptosis has also been shown to result in the malignant transformation of cells (Korsmeyer, 1992).

Individual proto-oncogenes that have been associated with apoptosis may be expressed in cells undergoing apoptosis, and modulation of expression of individual proto-oncogenes has been observed to affect the process. Exemplary proto-oncogenes include c-myc, Fas (APO-1), p53, and Bcl-2 in addition to other genes such as ced-3, ced-4, ced-9 and Ice (Stellar, 1995; Cohen, 1993).

A. The Role of PKR and TNF-α in Apoptosis

TNFs, as prototypes proinflammatory cytokines are cytotoxic proteins produced by activated immune cells during the processes of pathogen elimination, antiviral activities, and tumor destruction. However, high levels of TNF-alpha in vivo can be detrimental since TNF-alpha induces metabolic disturbances, wasting, and suppression of hematopoiesis. At the cellular level, TNF-alpha induces production of superoxide radicals, activation of lysosomal enzymes (Larrick, et al., 1990; Liddil, et al., 1989), and fragmentation of DNA by the activation of endonuclease activity (Rubin, et al., 1988), leading to apoptosis.

Various mechanism have been proposed for TNF-α-associated apoptosis (Dressler, et al., 1992; Obeid, et al., 1993). It has been shown that: (1) TNF-α treatment results in the activation of several serine/threonine protein kinases including PKR; (2) TNF-α and PKR mobilize NF-kB; (3) PKR plays a pivotal role in the TNF-α: signaling pathway; and (4) tumor suppressor gene p53 plays a role in the TNF-α-induced apoptosis process. (See, Guy, et al., 1992; Van Lint, et al., 1992, Yeung and Lau, et al., 1996).

IV. Suppression or Delay of Apoptosis

The invention provides methods for enhanced production of cytokines in mammalian cell culture by suppressing the apoptotic cell death process. By inhibiting apoptosis, the cell lines described herein have a longer lifespan in culture and exhibit an increase in biosynthesis of cytokines and/or the time over which the cells function to produce cytokines is increased.

In one aspect, the invention provides a method for modulating cytokine or other protein production by modifying the cells within a cell culture in a manner effective to result in the suppression or delay of apoptosis. As described herein, the inventors have observed that a cell line may be modified in a manner effective to result in the suppression or delay of apoptosis in conjunction with one or more of further modification, priming and induction, such that above-normal levels of cytokine or other protein production are achieved relative to a parental cell line cultured under the same conditions, which has not been so modified.

In another aspect, the invention provides a method of producing a cytokine or other protein, comprising culturing a host cell transfected with an expression vector having a promoter which functions in the host cell, operably linked to a DNA sequence encoding a protein the expression of which is effective to inhibit apoptosis.

Suppression of the apoptotic cell death process in mammalian cell culture may be achieved by any of a number of strategies directed to inhibition of apoptosis, including: (1) overexpression of a protein capable of inhibiting apoptosis, examples of which include, but are not limited to CrmA, Bcl-2a and Bcl-X_{L} or a homologue thereof; (2) suppression of eIF2-alpha (GenBank Accession No. A 457497) phosphorylation, e.g., by overexpression of a mutant form of eIF2-alpha or eukaryotic translation initiation factor (eIF-3), prepared by mutation of the respective endogenous gene using homologous recombination or site directed mutagenesis (thereby inhibiting the downstream substrates of PKR); (3) suppression of endogenous FADD activity, e.g., by overexpression of a mutant form of FADD, prepared by mutation of the endogenous FADD gene using homologous recombination or site directed mutagenesis; or (4) use of a transdominant mutant, by mutation of an endogenous gene for one or more pro-apoptotic counterparts of Bcl-2a, e.g. BAX (GenBank Accession No. L22473), BAK (GenBank Accession No. BE221666), and/or Bcl-X_{S} (GenBank Accession No. L20122) by homologous recombination or site-directed mutagenesis, or by gene ablation or gene deletion of one or more of BAX, BAK, and Bcl-X_{S}.

In one preferred aspect of the invention, a selected gene, e.g., CrmA, Bcl-2a, Bcl-X_{L} or a homologue thereof is overexpressed in the host cell resulting in a suppression or delay in apoptotic cell death. In other cases, suppression of endogenous gene expression may result in suppression or delay of apoptotic cell death and can be effected by methods including, but not limited to, mutation of the endogenous gene, homologous recombination or site directed mutagenesis, gene deletion or gene ablation or any method effective to result in the abolition or altered expression of the target gene.

Cell death may be detected by staining of cells with propidium iodide (PI), or by use of assays specific to apoptotic cell death, e.g., by staining with annexin V (Vermes, et al., 1995).

Necrotic cell death may be distinguished from apoptotic cell death by evaluating the results of a combination of the assays for cell viability, together with microscopic observation of the morphology of the relevant cells.

As set forth above, apoptosis may be inhibited by increasing the expression of proteins associated with blocking the apoptotic process in nature. Alternatively, apoptosis may be inhibited by decreasing the expression of proteins associated with facilitating the apoptotic process in nature, e.g., by modifying cells in a manner effective to express modified or variant forms of such proteins.

A. Enhancing Expression of Cytokine Response Modifier A (CrmA)

Poxviruses encode several cytokine response modifying (Crm) proteins, which possess sequence homology to a number of human proteins important to the immune response (Zhou Q, et al., 1997; Dbaibo GS et al., 1998). The cowpox virus cytokine response modifier A (CrmA) inhibits both serine and cysteine proteases (Tewari, M et al., 1995) and has been demonstrated to be a potent inhibitor of apoptosis induced by serum withdrawal (Nicholson, D. W et al., 1995), activation of the Fas or TNF-receptors (Kostura, M J et al., 1989; Chinnaiyan, A M et al., 1996), or withdrawal of nerve growth factor in primary chicken neuronal cultures (Orth, K et al., 1996). CrmA has been shown to have preferential activity as a competitive inhibitor against IL-1β converting enzyme (ICE) (Muzio, M et al., 1996), but also exhibits less potent inhibition of the proteolytic activity of other members of the ICE family implicated in apoptosis. (See, e.g., Dbaibo G S et al., 1998 and Dbaibo G S et al., 1997.)

Proteases belonging to the IL-1β converting enzyme (ICE) 1 family are considered to be central to the apoptotic process, partially based on the observations: that apoptosis is induced when these proteases are overexpressed in their active form (Miura, M et al., 1993); and apoptosis is inhibited when these proteases are specifically inhibited (Tewari, M et al., 1995).

The sphingolipid ceramide has recently been shown to be a potent inducer of apoptosis in a number of different systems. (See, e.g., Brenner B, et al., 1998 and Wegenknecht B et al., 1998. In addition, TNF-α, Fas ligation, serum withdrawal, some chemotherapeutic agents and γ-irradiation (all of which are reported to induce apoptosis) have been shown to elevate cellular levels of ceramide, suggesting that the biological activity of ceramide is common to a pathway shared by a variety of inducers of apoptosis (Jayadev, S, et al., 1995; Haimovitz-Friedman, A, et al., 1994; and Tepper, C. G, et al., 1995).

The apoptotic death signal from both Fas and the TNF-α receptor 1 have observed to operate by way of FADD, a “death domain” containing protein that belongs to a new family of signaling molecules that associate with members of the TNF-α receptor family. Expression of a dominant negative mutant of FADD was shown to inhibit ceramide accumulation, ICE-related protease activation, and apoptosis after treatment with Fas antibody. Exogenously provided ceramide was able to bypass this block and produce apoptosis (Chinnaiyan, A. M. et al., 1996).

Ceramide accumulation in MCF-7 cells after treatment with TNF-α was observed to be completely inhibited by CrmA, suggesting that CrmA targets apoptotic signaling upstream of ceramide generation in response to TNF-α. Exogenous ceramide was shown to bypass blocking by CrmA and produce apoptosis (Dbaibo et al., 1997).

In contrast, Bcl-2 protects from both TNF-α and ceramide-induced cell death without interfering with ceramide generation, suggesting that it functions further downstream along the ceramide pathway (Dbaibo et al., 1997).

B. Enhanced Expression of Bcl-2, Bcl-X_{L} or a Homologue Thereof

Members of the Bcl-2 family have been shown to act as either inhibitors or promoters of apoptosis. Bcl-2 was discovered as a gene the expression of which was increased by chromosomal translocations in B-cell malignancies (extensively reviewed by Reed). Bcl-2 has been found to be activated in the majority of follicular non-Hodgkin's lymphomas and less frequently in other malignancies such as prostate cancer. Its activation has also been seen in some benign conditions such as follicular hypertrophy of lymph nodes and tonsils.

In multiple types of cells including, but not limited to lymphocytes, fibroblasts, neurons and hematopoietic cells, expression of Bcl-2 has been shown to delay or even prevent apoptosis. Conversely, down-regulation of Bcl-2 in many of these systems has been shown to promote apoptosis. Bcl-2 is a membrane-associated protein typically found in the nuclear envelope, endoplasmic reticulum and mitochondria of the intact cell.

The Bcl-2 family of gene products is commonly involved in apoptotic processes. Bcl-2a and Bcl-X_{L} are considered to be anti-apoptotic proteins (Boise and Thompson, 1995; Schendel, 1998) and previous studies on lymphocytic and myeloid cells have indicated a role for Bcl-2a in the maintenance of cell growth and the prevention of cell death (Cohen, 1993). Additionally, Bcl-2a plays a significant role in prevention of neuronal cell apoptosis (Garcia, et al., 1992), probably by decreasing the generation of reactive oxygen species (Kane, et al., 1993).

The viability of many cells is dependent on a constant or intermittent supply of cytokines or growth factors. In the absence of such cytokines or growth factors, the cells undergo apoptosis. The Bcl-2 family of proteins are integral to the apoptotic process mediated by cytokines. Over-expression of Bcl-2 and Bcl-X_{L} has been shown to suppresses apoptosis when cytokines are withdrawn. Overexpression of BAX, and BAK has been shown to override the incoming signals from cytokine receptors and induce apoptosis.

In one exemplary application of the present invention, Bcl-2 overexpressing cells were produced by transfecting a target cell line with a pSV-2-Bcl2 expression plasmid (Reed, et al., 1988; Reed, et al., 1981). The Bcl-2 overexpressing cells were generated, selected and further cultured in a manner effective to result in production of cytokines and other proteins, then cultures were analyzed for cytokine or protein production, as further described below in Example 4.

C. Suppression of eIF2-alpha Phosphorylation

It has been demonstrated that PKR plays a critical role in TNF-induced and p53mediated apoptosis in cells including promonocytic U937 cells (Yeung, M. C., et al., 1996; Yeung, M., and Lau, A. S., 1998). Suppression of PKR activity, by transfecting U937 cells with PKR-antisense or PKR-mutant expression plasmids renders the cells more resistant to TNF or endotoxin induced cytotoxicity. Since eIF-2alpha is a physiological substrate of PKR, its phosphorylation by PKR has been shown to be sufficient to induce apoptosis.

Consistently, TNF-induced apoptosis has been correlated with increased phosphorylation of the alpha subunit of the eIF-2 (Srivastave, et al., 1998).

As set forth above, eIF-2alpha contributes to the inhibition of cellular and viral protein synthesis following phosphorylation. It follows that suppression of PKR-mediated phosphorylation of eIF-2alpha, by mutating the phosphorylation site of the factor, provides a means to inhibit the apoptotic affect of PKR overexpression on cultured cell lines.

A variant eIF-2 alpha protein was expressed in lymphoid cells, using a vector containing the coding sequence for a modified form of eIF-2 alpha under the control of a strong viral promoter. Cells expressing the modified form of eIF-2a were generated, selected, further cultured in manner effective to result in production of the cytokine or other protein of interest, and analyzed for the biosynthesis of the cytokine or other protein of interest (data not shown).

D. Suppression of Endogenous FADD Activity

The Fas receptor is a member of the TNF and the nerve growth factor receptor superfamily (Stellar, 1995). Following binding of Fas ligand to the Fas receptor, apoptosis is initiated via immediate downstream effectors, including FADD, FLICE, and TRADD. FADD is a cytoplasmic protein with a death domain which is crucial for CD 95 ligand and TNF induced apoptosis.

The binding of these proteins to their respective receptors results in activation of the caspase protease cascade and facilitates apoptosis. It has been previously demonstrated that Fas expression and consequent apoptosis are regulated by PKR activity in NIH-3T3 cells (Donze, et al., 1999). In cells transfected with a transdominant negative mutant deficient in PKR kinase activity, the expression of Fas, TNFR-1, FADD (Fas-associated death domain), FLICE, Bad and BAX are suppressed, and the cells were resistant to apoptosis-inducing agents. Additionally, murine fibroblasts lacking FADD were almost resistant to dsRNA-mediated cell death (Balachandran, et al., 1998).

Variant, non-functional human and murine FADD genes were generated from the wild type FADD gene (Chinnaiyen, et al., 1995; Yeh, et al., 1998). Mutant genes have been used to generate murine FADD−/− cells that were deficient in FADD activity with consequent resistance to PKR-mediated cytotoxicity (Balachandran, et al., 1998). The results suggest that the Fas-mediated cell death process is inhibited or eliminated in cells expressing a modified FADD gene, allowing for inhibition of apoptosis and that the inhibitory effect of this inactive form of FADD is not circumvented by PKR activation.

For use in practicing the present invention, a mutated FADD cDNA sequence was inserted into a vector effective to express the inserted fragment under the control of a strong viral promoter. Cells expressing a modified form of FADD were generated, selected, further cultured in manner effective to result in production of cytokines and analyzed for the biosynthesis of the cytokine or other protein of interest (data not shown).

E. Inhibiting Pro-Apoptotic Counterparts of Bcl-2

In general, BAX, BAK, Bcl-X_{S} and others are pro-apoptotic proteins (Boise and Thompson, 1998). Overexpression of BAX, BAK and Bcl-X_{S} has been shown to override the signals from cytokine-mediated signaling associated with cell viability and to induce apoptosis.

A mutated or variant human BAX, BAK, or Bcl-X_{S} cDNA sequence may be inserted into a vector effective to express the inserted fragment. Cells expressing a modified form of human BAX, BAK, or Bcl-X_{S} are thereby generated, selected, further cultured in manner effective to result in production of a cytokine or other protein of interest, and then analyzed for the biosynthesis of the cytokine or other protein of interest, as described below.

In one preferred embodiment a modified eIF-2a, FADD, Bcl-X_{S}, BAK or BAX protein for use in practicing the invention is a derivative or variant form of the respective protein as found in nature. That is, the derivative polypeptide or protein contains at least one amino acid substitution, deletion or insertion. The amino acid substitution, insertion or deletion may occur at any residue within the amino acid sequence of the polypeptide or protein, as long as it interferes with the biological activity of the protein.

Modified or variant forms of such native proteins are ordinarily prepared by site specific mutagenesis of one or more nucleotides in the nucleic acid sequence encoding the eIF-2a, FADD, Bcl-X_{S}, BAK or BAK protein, using cassette or PCR mutagenesis or another techniques known in the art to produce DNA encoding a modified or variant protein, and thereafter expressing the DNA in recombinant form in cell culture.

Site-specific mutagenesis provides a means for introducing one or more nucleotide sequence changes into the DNA encoding a given protein. The technique of site-specific mutagenesis is generally known in the art, and typically employs a phage vector which exists in both a single stranded and double stranded form.

It will be understood that the mutant, modified or variant forms of native proteins described herein can be created by point or site directed mutagenesis of the appropriate nucleic acid sequence, or by homologous recombination (knock-in or knock-out) to accomplish inhibition of function or activity of the target gene or the corresponding protein.

In one exemplary approach, the cDNA sequence for both a yeast and human form of the gene encoding a modified eIF-2a, FADD, Bcl-X_{S}, BAK or BAK protein, respectively, is inserted into an expression vector under the control of a promoter. Exemplary promoters include both constitutive promoters and inducible promoters, examples of which include a CMV promoter, an SV40 early promoter, an RSV promoter, an EF-1α promoter, a promoter containing the tet responsive element (TRE) in the tet-on or tet-off system as described (ClonTech and BASF), the beta actin promoter and the metallothienein promoter that can upregulated by addition of certain metal salts.

When a variant or mutant, non-functional human BAX, BAK or Bcl-X_{S} gene is incorporated into a heterologous nucleic acid construct and used to generate transformed cells deficient in BAX, BAK, or Bcl-X_{S} activity, respectively, apoptosis is inhibited. The inhibitory effect of such a biologically inactive form of BAX, BAK, or Bcl-X_{S} on apoptosis provides a means to circumvent the stimulatory effect of the overexpression of cytokine regulatory factor, such as PKR on apoptotic cell death in cultured cell lines.

V. Enhanced Cytokine Activity Expression or Production

A. Cytokine Regulatory Factor Overexpression

In one approach, increasing the expression of a cytokine regulatory factor in a mammalian cell is used to increase cytokine activity, expression or production. Mammalian cell lines that express a higher than normal constitutive level of one or more cytokine regulatory factors or in which cytokine regulatory factor expression can be induced to higher than normal level are therefore useful for the production of cytokines.

The cells used to produce a given cytokine can overexpress one or more cytokine regulatory factors, e.g., PKR, from any mammalian source.

For example, a vector comprising a PKR-encoding nucleic acid sequence may be introduced into a cell, resulting in overexpression of PKR by the cell. Exemplary coding sequences for use in such vectors include, but are not limited to the coding sequence from the human p68 PKR gene found at GenBank Accession No. M35663, the murine PKR gene, the form of PKR normally found in rabbit reticulocytes or human peripheral blood mononuclear and other eIF-2-alpha kinases including yeast GCN2 and hemin regulated inhibitor (Wek R C, 1994). In a preferred approach, the human p68 kinase form of PKR is overexpressed, in a human cell line.

In some cases, one or more of the cytokine regulatory factors overexpressed is a mutant, variant form or an analog of the native form of the cytokine regulatory factor, e.g., in the case of PKR, a non-natural protein kinase that can mediate dsRNA activation of cytokine and other protein transcription (usually obtained by modification of the gene encoding a native PKR protein). Upon expression, mutant or variant forms of a given cytokine regulatory factor may have increased or decreased activity.

In accordance with the present invention, it has been discovered that cell viability may be increased in cells that overexpress the exemplary cytokine regulatory factor, by inhibiting apoptosis in the cells, resulting in enhanced cytokine production. (See Examples 1 and 4) With particular regard to PKR, suppression of a PKR inhibitor, p53, has been demonstrated to result in enhanced PKR activity (Tan S L, et al., 1998). Alternatively, deprivation of serum and growth factors such as IL-3 may be used to induce PKR activity in the cells, or PKR expression may be enhanced by a regulatory factor that interacts with the promoter controlling the expression of a PKR-encoding nucleic acid sequence. In the case of expression of the endogenous PKR-encoding nucleic acid sequence, exemplary regulatory factors include the interferon-inducible GAS elements, the IL-6 sensitive NF-IL6 and APRF elements and NF-KB elements. (See, e.g., Jagus R. et al., 1999 and Williams B R, 1999.)

B. Generation of Cytokine Regulatory Factor Overexpressing Cell Lines

Cytokine regulatory factor overexpressing or overproducing cells may be obtained by: (i) limiting dilution cloning of a parental cell line capable of expressing one or more cytokine regulatory factors, screening for cytokine regulatory factor activity, expression and/or production, and selecting for subclones that exhibit at least a 2-fold (2×) increase in cytokine regulatory factor activity, expression and/or production; or (ii) modifying a parental cell line capable of expressing cytokine regulatory factor by introducing into the cells a cytokine regulatory factor-encoding nucleic acid sequence under conditions effective to result in at least a 2-fold (2×) increase in cytokine regulatory factor activity, expression and/or production.

It follows that PKR overexpressing or overproducing cells may be obtained by: (i) limiting dilution cloning of a parental cell line capable of expressing PKR, screening for PKR activity, expression and/or production, and selecting for subclones that exhibit at least a 2-fold (2×) increase in PKR activity, expression and/or production; or (ii) modifying a parental cell line capable of expressing PKR by introducing into the cells a PKR-encoding nucleic acid sequence under conditions effective to result in at least a 2-fold (2×) increase in PKR activity, expression and/or production, as further described in co-owned U.S. application Ser. No. 09/657,881, expressly incorporated by reference herein.

1. Increasing Endogenous Cytokine Regulatory Factor Activity, Expression and/or Production

In one embodiment, the invention provides a native cell line that overexpresses or overproduces an endogenous cytokine regulatory factor coding sequence and the use of such a cell line for the production of one or more cytokines.

In one preferred aspect of this embodiment, a native cell line that overexpresses or overproduces an endogenous cytokine regulatory factor is modified by transformation of the cells with an anti-apoptotic gene and/or priming and/or induction, to enhance cytokine production by the cells.

In practicing the method, a cell line capable of expressing a cytokine regulatory factor and one or more cytokines (referred to herein as a “parental cell line”) is identified and subjected to limiting dilution cloning of single cells, using standard methods routinely employed by those of skill in the art. In general, the subcloning step is carried out at least 3 times, preferably at least 5 times and typically from 5 to 10 times in 96 well plates. Subclones are grown to obtain a population of approximately 0.3 to 0.5 million cells/ml using culture conditions typically employed to culture the parental cell line. The subclones are then assayed for cytokine regulatory factor expression by evaluating transcription (mRNA) and/or protein levels (Western blot) and/or biological activity, using methods known in the art for the particular cytokine regulatory factor.

By way of example, assays for PKR activity include autophosphorylation assays (Der et al., 1995), an assay for eIF2α phosphorylation (Zamanian-Daryoush, et al., 1999), and a kinase assay (carried out by immunoprecipitation of PKR and in vitro assay for kinase (Zamanian-Daryoush, et al., 2000).

Exemplary assays generally applicable to the analysis of cytokine regulatory factor expression and/or production include, protein assays such as Western blot and assays for mRNA such as RT-PCR (reverse transcriptase polymerase chain reaction) and Northern blotting, dot blotting, or in situ hybridization using an appropriately labeled probe based on the cytokine regulatory factor-encoding nucleic acid sequence.

Subclones that exhibit a level of cytokine regulatory factor expression or production that is at least 2-fold (2×), and preferably 3-fold (3×), 4-fold (4×), 5-fold (5×) or more greater than the level of cytokine regulatory factor expression or production of the parental cell line are selected. In some cases, such selected subclones exhibit a level of cytokine regulatory factor expression or production that is 10-fold (10×) or more the level of expression or production of the parental cell line.

Selected subclones are then modified and/or treated in a manner effective to result in enhanced cytokine production. Modified generally means transformation of the cells with an anti-apoptotic gene, while treatment generally means priming and/or induction, to enhance cytokine production by the cells, as further detailed below.

2. Expression of a Cytokine Regulatory Factor-Encoding Nucleic Acid in a Host Cell

The invention also provides host cells which have been transduced, transformed or transfected with an expression vector comprising a cytokine regulatory factor-encoding nucleic acid sequence. The culture conditions, such as temperature, pH and the like, are those previously used for the parental cell line prior to transduction, transformation or transfection and will be apparent to those skilled in the art.

In one approach, a mammalian cell line is transfected with an expression vector having a promoter or biologically active promoter fragment or one or more (e.g., a series) of enhancers which functions in the host cell line, operably linked to a DNA segment encoding PKR, such that the PKR is overexpressed in the cell line.

In a preferred aspect of this approach, cells are first transfected with a vector containing an anti-apoptotic gene, selected and successful transformants further modified to produce a cytokine regulatory factor overexpressing cell line.

By overexpression of a cytokine regulatory factor is a meant higher than normal level of cytokine regulatory factor activity. Typically, “normal” cytokine regulatory factor activity or expression is reported as a range of cytokine regulatory factor activity or expression, which is generally observed for a corresponding parental cell line which has not been transfected with a vector encoding cytokine regulatory factor, has not been primed or induced and is uninfected. It will be understood that the range of normal cytokine regulatory factor activity for a given type of cells may vary somewhat dependent upon culture conditions.

Higher than normal cytokine regulatory factor expression means at least 150%, preferably at least 200%, 300% or 400%, and more preferably 500% or more of the normal cytokine regulatory factor level produced or expressed by the corresponding parental cell line. A cytokine regulatory factor-overexpressing cell line may be constitutive for cytokine regulatory factor overexpression or inducible for cytokine regulatory factor overexpression.

In one preferred approach, the cytokine regulatory factor-overexpressing cell line is inducible for cytokine regulatory factor overexpression in order to regulate the level of the cytokine regulatory factor available for cytokine induction.

C. Enhanced Cytokine production

While enhanced cytokine regulatory factor expression is described herein as a means to enhance cytokine activity, expression or production, it will be understood that it is not necessary to measure or directly increase cytokine regulatory factor expression in order to enhance cytokine activity, expression or production.

In one alternative approach, cytokine production may be increased with or without the insertion of a heterologous nucleic acid construct encoding either a cytokine regulatory factor (such as PKR, IRF-3 , IRF-7, NF-KB or another transcription factor), or a heterologous nucleic acid construct encoding an anti-apoptotic protein. In one example, this is accomplished by selecting for a cell line with enhanced expression of an anti-apoptotic protein, e.g., a cell line that expresses an enhanced level of CrmA, selected for by subcloning. Such a cell line may be modified by introduction of a heterologous nucleic acid construct encoding a cytokine regulatory factor, prior to or after selection by subcloning.

As detailed herein, cells treated by priming and/or induction using appropriate methodology exhibit an increase in cytokine production. In other words, the invention includes methods and compositions for enhanced cytokine activity, expression or production that do not include either selection or modification of the cells to enhance cytokine regulatory factor expression. However, it will be understood that such enhanced cytokine regulatory factor expression may take place when cells are primed and/or induced under appropriate conditions resulting in increased cytokine production.

Enhanced or “higher than normal” cytokine activity, production or expression means at least 150%, preferably at least 200%, 300% or 400%, and more preferably 500% or more of the level of cytokine activity, production or expression exhibited by the corresponding parental cell line. A cell line which exhibits enhanced or higher than normal cytokine activity, production or expression may be constitutive for cytokine expression or inducible for cytokine expression.

VI. Inhibition of Apoptosis and Enhanced Cytokine Expression and Production

Similarly, a cell line for use in practicing the invention is inducible for overexpression of a protein that interferes with the apoptotic process, in order to regulate the apoptosis in conjunction with cytokine regulatory factor expression for optimal cytokine production.

A. Inhibition of Apoptosis

In general, cytokine production may be increased by inhibiting apoptosis in a cell line characterized by overexpression of one or more cytokine regulatory factors and one or more cytokines.

In one preferred embodiment, the invention provides a cell line transfected with a first heterologous nucleic acid construct or expression vector effective to express the coding sequence for a protein capable of inhibiting apoptosis in the cells, under the control of a first promoter. Exemplary proteins capable of inhibiting apoptosis include, but are not limited to, CrmA, Bcl-2a, Bcl-X_{L}, a modified from of eukaryotic translation initiation factor 2 alpha (eIF-2 alpha) or eukaryotic translation initiation factor (eIF-3), a modified form of Fas-associated death domain (FADD), a modified form of Bcl-X_{S}, a modified form of Bcl-2-homologous antagonist/killer (BAK) and a modified from of BAX, such as Bcl-2a or Bcl-X_{L}.

In one aspect of this embodiment, the same cell line is modified in a manner effective (i) to express the coding sequence for a protein capable of inhibiting apoptosis (an “anti-apoptotic” protein); and (ii) to express the coding sequence for one or more cytokine regulatory factors, e.g., PKR. In general, the coding sequences are introduced into the cells by way of separate heterologous nucleic acid constructs. For example, a first heterologous nucleic acid construct or expression vector will typically comprise the coding sequence for an anti-apoptotic protein, e.g., CrmA, a first promoter, and a first selectable marker-encoding nucleic acid sequence. Similarly, a second heterologous nucleic acid construct or expression vector will typically comprise the coding sequence for the coding sequence for one or more cytokine regulatory factors, e.g., PKR, a second promoter, and a second selectable marker-encoding nucleic acid sequence. However, both coding sequences may be introduced into cells using a single vector.

In practicing this aspect of the invention, the cells may be (1) transfected with a single vector comprising a first coding sequence encoding an anti-apoptotic protein, a first promoter, a first selectable marker-encoding nucleic acid sequence and a second coding sequence encoding a cytokine regulatory factor, a second promoter and a second selectable marker-encoding nucleic acid sequence; (2) transfected with a first and second expression vector (as described above) at the same time; (3) transfected with a first expression vector, a stable transgenic cell line selected for, then the selected cells transfected with a second expression vector and double transformants selected for; (4) transfected with a first expression vector only with a stable transgenic cell line selected for; or (5) transfected with the second expression vector, a stable transgenic cell line selected for, then the selected cells transfected with the first expression vector and double transformants selected for.

In the preferred approach, a stable transgenic cell line prepared as described in any one of (1)-(5) above, is primed and/or induced to further enhance cytokine production, as further described below.

B. Treatment Of Cells To Further Enhance Cytokine Production

In another aspect of the invention a cell line that expresses a protein effective to inhibit apoptosis is primed and/or treated (induced) in a manner effective to result in an increase in cytokine production.

The cell line may be modified in a manner effective to inhibit apoptosis prior to, or after the cell line is subjected to one or more of selection, modification, priming and treatment (induction) in a manner effective to result in increased cytokine production.

In one preferred approach, the method comprises: (a) modifying a cell line capable of cytokine production in a manner effective to inhibit apoptosis, by introducing a heterologous nucleic acid construct comprising the coding sequence for an anti-apoptotic protein, e.g. a gene encoding CrmA into the cells of the cell line, and further modifying the cell line by introducing an exogenous cytokine regulatory factor-encoding nucleic acid sequence into the cell in a manner effective to express the factor and growing the cells to produce a cytokine regulatory factor overexpressing cell line which also expresses an anti-apoptotic gene; or (b) selecting for cells that overexpress an endogenous cytokine regulatory factor-encoding nucleic acid sequence and growing the cells to produce a cytokine regulatory factor overexpressing cell line and further modifying the cell line in a manner effective to inhibit apoptosis, by introducing a heterologous nucleic acid construct comprising the coding sequence for an anti-apoptotic protein, e.g. a gene encoding CrmA into the cells of the cell line to produce a cytokine regulatory factor overexpressing cell line which also expresses the anti-apoptotic gene; or (d) selecting for cells which exhibit a higher level of expression of an endogenous anti-apoptosis gene, such as a CrmA homologue; or (e) selecting for cells which exhibit a higher level of expression of an endogenous anti-apoptosis gene, such as a CrmA homologue and further modifying the cell line by introducing an exogenous cytokine regulatory factor-encoding nucleic acid sequence into the cell in a manner effective to express the factor and growing the cells to produce a cytokine regulatory factor overexpressing cell line which also expresses the anti-apoptotic gene; and/or (f) priming and/or induction.

Priming is a well known phenomenon whereby pretreatment of cells with a priming agent results in enhanced production of one or more cytokines, when applied in conjunction with treatment or induction. Exemplary priming agents include, but are not limited to phorbol myristate acetate (PMA) and other phorbol esters, calcium ionophores, interferon-α, interferon-γ, interferon-β, G-CSF, GM-CSF, PDGF, TGF, EGF or chemokines (IL-8, MCP or MIP), sodium butyrate, endotoxin, a kinase activator (e.g., protein activator of PKR, PACT), or a transcription activator (NF-KB, IRFs including IRF-3 and IRF-7). Suitable priming agent concentrations may be found in the scientific literature, e.g., a concentration of PMA in the range 5-50 nM, preferably about 10 nM, is suitable.

Induction or treatment refers to the addition of a microbial (e.g., viral, bacterial, or fungal) inducer, an extract of a microbe capable of acting as an inducer (e.g., an endotoxin or bacterial cell wall containing extract), or a non-microbial inducer to the cell culture.

Exemplary methods of non-microbial induction include, but are not limited to, exposure to double-stranded RNA (dsRNA) such as poly(I):poly(C) or poly r(I):poly r(C) (poly IC); exposure to small molecules, e.g., polyanions, heparin dextran sulfate, chondroitin sulfate, cycloheximide, Actinomycin D, calcium ionophores or sodium butyrate and exposure to cytokines.

Exemplary methods of viral induction include, but are not limited to, (1) exposure to live virus, e.g., Sendai virus, encephalomyocarditis virus or Herpes simplex virus; (2) exposure to the aforementioned killed virus; or (3) exposure to isolated double-stranded viral RNA. In addition, cytokine induction may be produced or enhanced by adding particular cytokines known to stimulate cytokine production in certain cells.

After addition of the inducing agent, typically, cells are further incubated until desired levels of induced and secreted cytokines are obtained. Incubation at 37° C. for at least 12-48 hours, and up to 72-96 hours is generally sufficient.

In one exemplary application of the method, cells are primed with IFN-beta for approximately 24 hr, followed by exposure to medium containing polyI:C and cycloheximide for approximately 5 hrs, with Actinomycin D added during the last hour to a final concentration.

In another exemplary application of the method, cells are primed with IFN-beta for approximately 24 hr, then induced by treatment with a viral inducer, e.g., Sendai Virus (SV) for approximately 1 hr, followed by exposure to medium containing polyI:C and cycloheximide for approximately 5 hrs, with Actinomycin D added during the last hour to a final concentration.

Example 1 describes production of a CrmA expressing cell line, superinduction and viral induction of the CrmA expressing cell line. Example 2 describes exemplary vectors, transfection methods and production of a cytokine regulatory factor overexpressing cell line that also expresses the anti-apoptotic protein, Bcl-X_{L}, suitable for use in practicing the invention. Example 3 describes cytokine production by an exemplary cytokine regulatory factor overexpressing cell line and a cytokine regulatory factor overexpressing cell line that also expresses an anti-apoptotic protein.

VII. Expression Vectors and Transformation of Host Cells

A. Expression Vectors

By way of example, heterologous nucleic acid constructs or expression vectors were prepared for the generation of transgenic cell lines which express CrmA, Bcl-X_{L} and PKR. (See Examples 1 and 2). In particular, it is well known in the art of vector construction to obtain suitable plasmids or other vectors from commercial sources which are capable of introduction into and replication within selected human cells, where the plasmids may also be equipped with selectable markers, insertion sites, and suitable control elements, such as termination sequences. The plasmid may or may not have its own promoter or the promoter may be exchanged in a standard vector.

Heterologous nucleic acid constructs or expression vectors for use in practicing the invention include the coding sequence for an anti-apoptotic protein alone or in combination with the coding sequence for a cytokine regulatory factor. It will be understood that the term “antiapoptotic protein” may refer to a protein which directly inhibits apoptosis (e.g. CrmA, Bcl-2a or Bcl-X_{L}) or the modified form of a protein associated with apoptosis (e.g., a modified from of: eukaryotic eIF-2 alpha, eIF-3, FADD, Bcl-X_{S}, BAK or BAX, such as Bcl-2a or Bcl-X_{L}).

Variant forms of such coding sequences, fragments and splice variants thereof are included within the scope of the invention. In addition, the vector may include the coding sequence in isolation or in combination with additional coding sequences, such as a fusion protein or signal peptide coding sequence.

Standard methods for cutting, ligating and bacterial transformation, known to those of skill in the art are used in constructing vectors for use in practicing the present invention. See generally, Maniatis, et al., 1989; Ausubel, F. M., et al., 1993; and Gelvin, S. B., et al., 1990, all of which are expressly incorporated by reference, herein. The vectors and methods disclosed herein are suitable for the expression of the coding sequence for an anti-apoptotic protein and cytokine regulatory factor. Any vector may be used so long as it is replicable and viable in the mammalian cells into which it is introduced. Large numbers of suitable vectors and promoters are known to those of skill in the art, and are commercially available. Appropriate cloning and expression vectors for use in human cells are also described in Sambrook et al., 1989, and Ausubel F M et al., 1989, expressly incorporated by reference herein. The appropriate DNA sequence may be inserted into a plasmid or vector (collectively referred to herein as “vectors”) by a variety of procedures. In general, the DNA sequence is inserted into an appropriate restriction endonuclease site(s) by standard procedures. Such procedures and related sub-cloning procedures are deemed to be within the scope of knowledge of those skilled in the art.

The present invention relies on the use of heterologous nucleic acid constructs comprising one or more of the nucleic acid coding sequences described above. The constructs comprise a vector, such as a plasmid or viral vector, into which a sequence of the invention has been inserted, in a forward or reverse orientation.

Vectors are typically equipped with selectable markers, insertion sites, and suitable control elements, such as termination sequences. The vector may comprise regulatory sequences, including, for example, non-coding sequences, such as introns and control elements, i.e., promoter and terminator elements or 5′ and/or 3′ untranslated regions, effective for expression of the coding sequence in host cells (and/or in a vector or host cell environment in which a modified soluble protein antigen coding sequence is not normally expressed), operably linked to the coding sequence.

The promoter may be constitutive or inducible and may be a naturally occurring, engineered or hybrid promoter. Exemplary promoters include both constitutive promoters and inducible promoters, examples of which are a CMV promoter, an SV40 early promoter, an RSV promoter, an EF-1α promoter, a promoter containing the tet responsive element (TRE) in the tet-on or tet-off system as described (ClonTech and BASF), the beta actin promoter and the metallothienein promoter which can upregulated by addition of certain metal salts. Large numbers of suitable vectors and promoters are known to those of skill in the art, are commercially available and are described in Sambrook, et al., (supra).

Selectable markers for use in such expression vectors are generally known in the art and the choice of the proper selectable marker will depend on the host cell. Examples of selectable marker genes encode proteins that confer resistance to antibiotics or other toxins include ampicillin, methotrexate, tetracycline, neomycin (Southern and Berg, J., 1982), mycophenolic acid (Mulligan and Berg, 1980), puromycin, zeomycin, or hygromycin (Sugden et al., 1985).

Cells are transfected using standard procedures including electroporation, calcium phosphate, DEAE dextran, lipofection, or Lipofectamine treatment, and selected in the appropriate antibiotic. Procedures for the cloning and expression of modified forms of native protein using recombinant DNA technology are generally known in the art, as described in Ausubel, et al., 1992 and Sambrook, et al., 1989, expressly incorporated by reference, herein.

B. Nucleic Acid Coding Sequences

In accordance with the present invention, polynucleotide sequences which encode a given cytokine regulatory factor (CRF) or anti-apoptotic protein (which includes modified forms of a protein associated with apoptosis) includes splice variants, fragments, fusion proteins, modified forms of the proteins or functional equivalents thereof, collectively referred to herein as “CRF- or anti-apoptotic protein-encoding nucleic acid sequences”.

Due to the inherent degeneracy of the genetic code, other nucleic acid sequences which encode substantially the same or a functionally equivalent amino acid sequence may be used to clone and express the CRF- or anti-apoptotic protein-encoding nucleic acid sequences. Thus, for a given CRF- or anti-apoptotic protein-encoding nucleic acid sequence, it is appreciated that as a result of the degeneracy of the genetic code, a number of coding sequences can be produced that encode the same amino acid sequence. For example, the triplet CGT encodes the amino acid arginine. Arginine is alternatively encoded by CGA, CGC, CGG, AGA, and AGG. Therefore it is appreciated that such substitutions in the coding region fall within the sequence variants covered by the present invention. Any and all of these sequence variants can be utilized in the same way as described herein for the native form of a CRF- or anti-apoptotic protein-encoding nucleic acid sequence.

A “variant” CRF- or anti-apoptotic protein-encoding nucleic acid sequence may encode a “variant” CRF- or anti-apoptotic amino acid sequence which is altered by one or more amino acids from the native polypeptide sequence, both of which are included within the scope of the invention. Similarly, the term “modified form of”, relative to a CRF- or anti-apoptotic protein, means a derivative or variant form of the native CRF- or anti-apoptotic protein-encoding nucleic acid sequence or the native CRF- or anti-apoptotic amino acid sequence. Typically, a “modified form of” a native CRF- or anti-apoptotic protein or the coding sequence for the protein has a derivative sequence containing at least one amino acid or nucleic acid substitution, deletion or insertion, respectively.

Similarly, the polynucleotides for use in practicing the invention include sequences which encode native CRF- or anti-apoptotic proteins and splice variants thereof, sequences complementary to the protein coding sequence, and novel fragments of CRF- or anti-apoptotic protein encoding polynucleotides. The polynucleotides may be in the form of RNA or in the form of DNA, and include messenger RNA, synthetic RNA and DNA, cDNA and genomic DNA. The DNA may be double-stranded or single-stranded and if single-stranded may be the coding strand or the non-coding (antisense, complementary) strand.

As will be understood by those of skill in the art, in some cases it may be advantageous to produce nucleotide sequences possessing non-naturally occurring codons. Codons preferred by a particular eukaryotic host (Murray, E. et al., 1989) can be selected, for example, to increase the rate of CRF- or anti-apoptotic protein expression or to produce recombinant RNA transcripts having desirable properties, such as a longer half-life, than transcripts produced from the naturally occurring sequence.

A native CRF- or anti-apoptotic protein-encoding nucleotide sequence may be engineered in order to alter the coding sequence for a variety of reasons, including but not limited to, alterations which modify the cloning, processing and/or expression of the CRF- or anti-apoptotic protein by a cell.

1. Cytokine Regulatory Factors and Anti-Apoptotic Proteins

In one approach, a heterologous nucleic acid construct or expression vector for use in practicing the invention includes the coding sequence for a protein the active form of which is desired such as the coding sequence for a cytokine regulatory factor (CRF), exemplified herein by PKR or the coding sequence for an anti-apoptotic protein, exemplified herein by CrmA, Bcl-2 or Bcl-X_{L}.

In one general embodiment of the invention, a CRF or anti-apoptotic protein-encoding nucleic acid sequence has at least 70%, preferably 80%, 85%, 90% or 95% or more sequence identity to the native coding sequence found in GenBank. For example, a coding sequence useful for expression of human PKR has at least 70%, preferably 80%, 85%, 90% or 95% or more sequence identity to the sequence found at GenBank Accession No. M35663.

In the case of a cytokine regulatory factor or anti-apoptotic protein-encoding nucleic acid sequence, the substitution, insertion or deletion may occur at any residue within the sequence, as long as the encoded amino acid sequence maintains the biological activity of the native cytokine regulatory factor or anti-apoptotic protein.

B. Selection of Cell Lines

Any of a number of known cell types capable of producing one or more cytokines or proteins of interest may be employed in the methods of the invention. In practicing the invention, the selected cell line is modified in a manner to express a cytokine regulatory factor and/or anti-apoptotic protein-encoding nucleic acid sequence. Mammalian cell lines capable of cytokine or other protein production may be obtained by a number of methods known in the art, including isolation of primary cell lines or obtaining an established cell line from a commercial source.

Thus, the present invention provides a cell line comprising cells which have subjected to one or more of selection, modification, priming and induction effective to result in enhanced cytokine production or expression relative to the corresponding parental cell line.

Exemplary cell lines suitable for use in practicing the invention include, but are not limited to fibroblasts or immune cells, B cells (e.g., Namalwa, 293, Raji), monocytic cells (e.g., U937, THP-1), T cells, neutrophils, natural killer cells, MRC-5 cells, WI-38 cells, Flow 1000 cells, Flow 4000 cells, FS-4, FS-7 cells, MG-63 cells, CCRF-SB cells, CCRF-CEM, Jurkat cells, WIL2 cells and T98G cells.

Human cells are preferred for practicing the invention. In one exemplary application of the invention, a human cell line, e.g., Namalwa, is modified in manner effective to inhibit apoptosis and further modified, primed and/or treated in manner effective to result in enhanced cytokine or other protein production. Alternatively, the cells are modified in manner effective to inhibit apoptosis and selected for cytokine regulatory factor expression by limiting dilution subcloning to obtain cytokine regulatory factor-expressing subclones, which are then primed and/or treated in manner effective to result in enhanced cytokine or other protein production.

Examples of appropriate primary cell types which may be used in practicing the invention include, but are not limited to, cells of the monocyte/macrophage lineage, lymphocytic lineage cells including T- and B-cells, mast cells, fibroblasts, bone marrow cells, keratinocytes, osteoblast derived cells, melanocytes, endothelial cells, platelets, various other immune system cells, lung epithelial cells, pancreatic parenchmal cells, glial cells and tumor cells derived from such cell types. Modified, primed and/or induced cells are cultured under conditions employed to culture the parental cell line.

Generally, cells are cultured in a standard medium containing physiological salts and nutrients, such as standard RPMI, MEM, IMEM or DMEM, typically supplemented with 5-10% serum, such as fetal bovine serum. Culture conditions are also standard, e.g., cultures are incubated at 37° C. in stationary or roller cultures until desired levels of cytokine expression or production are achieved. Culturing the cells under conditions effective to facilitate recovery of cytokines include, but are not limited to culture in serum and/or protein-free or serum-free medium.

Preferred culture conditions for a given cell line may be found in the scientific literature and/or from the source of the cell line such as the American Type Culture Collection (ATCC; “http://www.atcc.org/”). Preferred culture conditions for primary cell lines, such as fibroblasts, B-cells, T-cells, endothelial cells, dendritic cells, and monocytes are generally available in the scientific literature.

In a further application of the invention, cells treated to inhibit apoptosis include cell lines generally used to express a given recombinant cytokine or protein of interest, wherein the expression of the cytokine or protein of interest is not associated with a cytokine regulatory factor such as PKR, e.g., CHO (Chinese hamster ovary) cells.

VIII. Cytokines

Cytokines elicit their biological activities by binding to their cognate receptors followed by signal transduction leading to stimulation of various biochemical processes. In some cases, the expression of such receptors is regulated by specific signals, e.g. a cytokine may be involved in positive or negative feedback loops and thereby regulate the expression of the receptor for the same or a different cytokine. Such receptors may be the same type of cell that produces the cytokine or a different type of cell.

Cytokines serve to mediate and regulate immune and inflammatory responses. In general, cytokine production is transient and production takes place during a short period of transcription resulting in production of mRNA transcripts which are also short-lived and subjected to post-transcriptional control mechanisms. Recent studies have indicated that a common signal transduction pathway, the “Jak/STAT” pathway, is used by a variety of cytokines (Abbas, et al., 1997).

It will be appreciated that the cellular source of cytokines is a distinguishing characteristic of each individual cytokine that may be produced by multiple diverse types of cells. In addition, a given cytokine (1) may act on more than one type of cells, (2) may have more than one effect on the same cell, (3) may have an activity shared with another cytokine, and (4) may influence the synthesis or effect of other cytokines, e.g., by antagonizing, or synergizing the effects thereof.

The cytokine(s) produced may be one or more of the following: interferons, including IFN-gamma, IFN-alpha and IFN-beta; tumor necrosis factors (TNF), including TNF-alpha, TNF-beta and TNF soluble receptors (sTNF-R); interleukins (IL), including IL-2, IL-3, IL-4, IL-5, IL-6, IL-7, IL-8, IL-11 and IL-12; colony stimulating factors, including granulocyte colony stimulating factors (G-CSF) and granulocyte-macrophage colony stimulating factor (GM-CSF); angiogenic factors, including fibroblast growth factor (FGF), vascular endothelial growth factor (VEGF); platelet-derived growth factors 1 and 2 (PDGF 1 and 2); chemokines, including Regulated Upon Activation Normally T-Expressed Secreted (RANTES); macrophage inflammatory proteins (MIP), such as MIP-1 alpha and MIP-2alpha; monocyte chemotactic protein-1 (MCP); anti-angiogenic factors, including angiostatin; endostatin leukemia inhibitory factor (LIF); ciliary neurotrophic factor; cardiotrophin and oncostatins, including oncostatin M.

The methods of the invention may also be used to increase the expression of any of a number of proteins which are capable of production in cell culture. Exemplary proteins include, but are not limited to, insulin, erythropoietin (EPO), tissue plasminogen activator (TPA), growth hormone and Factor VIII.

One exemplary group of cytokines, the interferons (IFNs) are produced in response to viral infection or growth of tumor cells. These glycoproteins possess anti-tumor and immunomodulatory activities in addition to their antiviral effects. Since 1994, IFNs have received FDA approval for specific clinical indications in the United States. Recently, two preparations of IFN-beta, one produced in E. coli and the other in Chinese hamster ovarian (CHO) cells, have been approved for patients with multiple sclerosis. The CHO cell-produced product has been shown to induce anti-IFN antibodies, and the formation of interferon immune complexes, in addition to causing undesirable effects such as injection site tissue necrosis in most patients. Additional deficiencies have been attributed to bacterially-produced IFNs, including the induction of antibodies and limited efficacy of IFN-alpha in various diseases, which may be attributed, in part, to a lack of all subtypes in the recombinant formulation. Previous studies have shown that the incidence of rejection as reflected by antibody formation can be as high as 20 to 38% for bacterially-produced IFN compared to only 1.2% for natural IFN-alpha (Antonelli, et al., 1991; Antonelli, et al., 1997).

The present invention is directed to providing improved cell line-produced cytokine compositions that lack undesirable side effects such as induction of an immune response when a administered to a patient or limited efficacy due to improper glycosylation or a lack of the full complement of native subtypes in the recombinant formulation.

The methods described herein are effective to result in enhanced cytokine production. In one preferred aspect of the invention, a combination of one or more of cell line modification, culture conditions, priming and inducing results in a significantly increase in cytokine production, e.g., an increase that represents at least 200% (2-fold or 2×), 250% (2.5-fold or 2.5×), 300% (3-fold or 3×), 400% (4-fold or 4×), 500% (5-fold or 5×), and preferably 1000% (10-fold or 10×) or more cytokine production or expression relative to the level exhibited by the same cell line under the same culture conditions absent modification, treating, priming or inducing the cells as described herein. In some cases, the methods of the invention result in an increase in cytokine production that is 100-fold (100×) to 1000-fold (1000×) or more.

IX. Evaluation of Cytokine or Other Protein Production, Isolation and Purification of Cytokines

A. Evaluation Cytokine or Other Protein Production

In order to evaluate the expression of a cytokine or other protein of interest by a cell line that has been subjected to one or more of modification, priming and/or induction, assays can be carried out at the protein level, the RNA level or by use of functional bioassays particular to the individual cytokine or other protein being expressed.

By way of example, the production and/or expression of a given cytokine may be measured in a sample directly, for example, by assays for cytokine activity, expression and/or production. Such assays include Northern blotting to quantitate the transcription of mRNA, dot blotting (DNA or RNA analysis), RT-PCR (reverse transcriptase polymerase chain reaction), or in situ hybridization, using an appropriately labeled probe (based on the cytokine-encoding nucleic acid sequence) and conventional Southern blotting.

Alternatively, protein expression, may be evaluated by immunological methods, such as immunohistochemical staining of cells, tissue sections or immunoassay of tissue culture medium, e.g., by Western blot or ELISA. Such immunoassays can be used to qualitatively and quantitatively evaluate expression of a cytokine or other protein. The details of such methods are known to those of skill in the art and many reagents for practicing such methods are commercially available.

A purified form of the cytokine or other protein is typically used to produce either monoclonal or polyclonal antibodies specific to the expressed protein for use in various immunoassays. (See, e.g., Harlow et al., 1988). Exemplary assays include ELISA, competitive immunoassays, radioimmunoassays, Western blot, indirect immunofluorescent assays and the like. In general, commercially available antibodies and/or kits may be used for the quantitative immunoassay of the expression level of known cytokines or other proteins, as exemplified in the analysis of interferon-beta in Example 1 and interferon-alpha in Example 4.

B. Isolation And Purification Of Cytokines

In general, cytokines produced in cell culture are secreted into the medium and may be purified or isolated, e.g., by removing unwanted components from the cell culture medium. Typically, the cytokines are fractionated to segregate cytokines having selected properties, such as binding affinity to particular binding agents, e.g., antibodies or receptors; or which have a selected molecular weight range, or range of isoelectric points.

Once increased production of a given cytokine or other protein is achieved, the cytokine or other protein thereby produced is purified from the cell culture. Exemplary procedures suitable for such purification include the following: antibody-affinity column chromatography, ion exchange chromatography; ethanol precipitation; reverse phase HPLC; chromatography on silica or on a cation-exchange resin such as DEAE; chromatofocusing; SDS-PAGE; ammonium sulfate precipitation; and gel filtration using, e.g., Sephadex G-75. Various methods of protein purification may be employed and such methods are known in the art and described e.g. in Deutscher, 1990; Scopes, 1982. The purification step(s) selected will depend, e.g., on the nature of the production process used and the particular cytokine or protein produced.

Specific examples are described above, however, it will be apparent to one of ordinary skill in the art that many modifications are possible and that the examples are provided for purposes of illustration only and do not limit the invention, unless so specified.

All patent and literature references cited in the present specification are hereby incorporated by reference in their entirety.

### EXAMPLE 1

Preparation and Characterization of a Transgenic CrmA-expressing Cell Line

A. Preparation of a Transgenic CrmA-Expressing Cell Line

The pEF FLAG-crmA-puro expression vector was constructed by inserting the coding sequence for CrmA in-frame into the pEF Bos vector described by Mizushima and Negata (NAR 18, 5322, 1990), based on the vector described by Huang et al., 1997. pEF FLAG-crmA-puro contains a full length cDNA encoding the anti-apoptotic CrmA protein (GenBank Accession No. Ml 4217; Cowpox virus white-pock variant (CPV-W2) (CrmA) gene, complete coding sequence) under the control of the strong elongation factor 1 alpha (EF-1 alpha) promoter and the puromycin resistance gene under the control of the pGK promoter. An additional feature of note is the coding sequence for the N-terminal FLAG epitope (Hopp et al., 1988) that was added to the CrmA nucleic acid sequence to facilitate detection of cell lines that express CrmA.

The vector also includes (i) a polyadenylation signal and transcription termination sequence to enhance mRNA stability; (ii) an SV40 origin for episomal replication and simple vector rescue; (iii) an ampicillin resistance gene and a ColE1 origin for selection and maintenance in E. coli; and (iv) a puromycin resistance marker (Puro) to allow for selection and identification of plasmid-containing eukaryotic cells after transfection with pEF FLAG-crmA-puro.

The one day before transfection MG-63 cells were seeded in a 6 well plate at 5×10⁴ per well. 2 μg of pEF FLAG-crmA-puro plasmid DNA was suspended in 100 μl Opti-MEM medium lacking serum, proteins or antibiotics. Lipofectamine reagent (Gibco, 10 μl) was diluted to 100 μl with Opti-MEM serum-free medium. Following gentle mixing of the two solutions, the mixture was incubated at room temperature for 45 min to allow for DNA-liposome complex formation. Immediately prior to treatment of MG-63 cells, 600 μl of Opti-MEM serum-free medium was added to the reaction tube containing the DNA-liposome mixture to obtain the final transfection solution. The cells were washed with PBS and followed by addition of the final DNA-liposome mixture and incubation for 4 hours at 37° C. This was followed by the addition of 1 ml MEM-5% FBS and incubation for an additional 16 hrs. The culture supernatant was removed by gentle aspiration and fresh cell growth medium (MEM supplemented with 5% FBS) added. After incubation for 48 hr, fresh media (MEM with 5% FBS) containing the selection marker, geneticin (G418, 500 μg/ml), was added to select for stable transfectants using standard methodology known in the art. In summary, a bulk population of stable transformants was obtained by selection with 500 μg/ml G418 (Gibco-BRL) for 3-4 weeks.

B. Characterization of a Transgenic CrmA-Expressing Cell Line

1. Increased Cell Viability

Wild type (WT) and CrmA-expressing (CrmA-#2) MG-63 cells were treated by Sendai virus (SV) induction and superinduction (SI; Inoue I et al., 1991) using the following procedure.

Cells were seeded at a cell density 2.5×10⁴ cells per well in 24 well plates, followed by incubation at 37° C. with CO₂ concentration at 5%. Following incubation, cells were primed with IFN-beta (100 IU/ml) for 24 hr. The cells were then induced by the addition of 1000 hemagglutinin units of SV in 200 μl of MEM medium supplemented with 2% fetal bovine serum (FBS) to each well, and incubation for one hour, followed by the addition of 300 μl of fresh medium containing polyI:C (100 μg/ml) and cycloheximide (5 μg/ml) and incubation for an additional 5 hrs. Actinomycin D was added during the last hour to a final concentration 4 μg/ml. After the induction process, the treated cells were washed 3 times with PBS to remove all inducers and resuspended in fresh MEM containing 2% FBS.

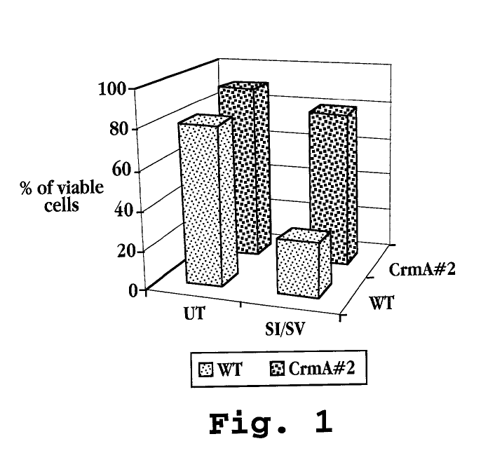

Wild type (WT) and CrmA-expressing (CrmA-#2) MG-63 cells that were not treated by Sendai virus (SV)± or superinduction (SI) were used as controls (UT). The viability of each type of cells was measured using a standard propidium iodide FACS assay. As shown in FIG. 1, CrmA expression inhibits SV/SI-induced cell death, indicated by a viability of up to 80% for CrmA-expressing cells at 20h after SV induction and SI treatment. In contrast, only 20% of wild type MG-63 cells exposed to the same conditions survived the process.

2. Enhanced Production of Cytokines

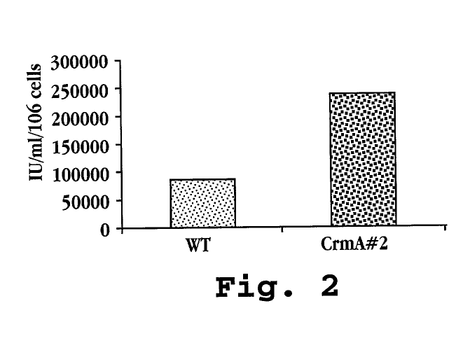

The cells were incubated for 20 hrs, then the culture medium from each well was collected and assayed for Interferon-beta (IFN-beta) production by ELISA. The IFN-beta ELISA was performed as described by the supplier. (Human Interferon-beta ELISA kit; distributed by TFB, Inc., and manufactured by FUJIREBIO, Inc., Tokyo, Japan). As shown in FIG. 2, there was significantly more IFN-beta produced by the CrmA#2 MG-63 cells, as compared to the MG-63 wild type counterparts.

CrmA-expressing (CrmA-#2) MG-63 cells were subjected to superinduction (SI) treatment in medium containing 0, 2mM, 4mM, and 8 mM of the nucleoside analog 2-aminopurine (2-AP), a known inhibitor of PKR.

SI (superinduction) treatment was carried out by seeding cells at a density of 2.5×10⁴ cells per well in 24 well plates at 37° C. at a CO₂ concentration of 5% the day before priming. Following incubation, the cells were primed with IFN-beta (100 IU/ml) for 24 hr, then 500 μl of fresh medium containing polyI:C (100 μg/ml) and cycloheximide (5 μg/ml) was added and the cells were incubated for an additional 5 hrs, with Actinomycin D added during the last hour to a final concentration 4 μg/ml. After the induction process, the treated cells were washed 3 times with PBS to remove all inducers and resuspended in fresh MEM containing 2% FBS.

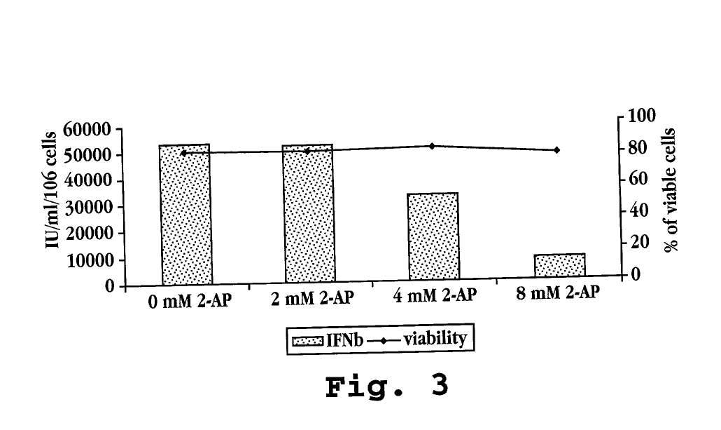

As shown in FIG. 3, 2-AP inhibited IFN-beta production in a dose-dependent manner, confirming that PKR plays a role in regulating IFN-beta expression.

3. Analysis of Flag-CrmA Protein Expression by Western Blot

Cells of the parental wild type cell line (MG-63-WT) and CrmA transformants (MG-63CrmA-#2) prepared as set forth above, were cultured to 100% confluence in 100 mm dishes. Cells were washed in cold phosphate buffered saline (PBS) and collected in a 1.5 ml microcentrifuge tubes using a cell scraper. Following further washings with PBS, the cells were incubated in lysis buffer (10 mM Tris-HCL [pH 7.5], 1% Triton X-100, 0.25% SDS, 50 mM KCL, 1 mM dithiothreitol, 2mM MgCl₂ and 1× Protein inhibitors cocktail [Roche]) for 10 min on ice, then centrifuged at 10,000g for 10 min. The lysate supernatant was transferred to a new microcentrifuge tube and the protein concentration measured using a BRL kit following the protocol provided by the manufacturer.

Cell lysates containing 100 μg of protein were loaded on a 4-12% NuPAGE Bis-Tris MOPS gel and subjected to electrophorectic separation, after which the gel was blotted onto a PVDF membrane. The membrane was further blotted in 5% milk-PBS overnight and exposed to primary rat anti-Flag antibodies, kindly provided by Dr. A Strasser (Royal Melbourne Hospital, Victoria, Australia) at dilutions of 1:500 for 1 hour. The blotted membrane was washed 3 times with PBS-0. 1% Tween-20 and incubated with secondary anti-rat-HRP-conjugated antibodies (1:2000) for 1 hour. The presence of the Flag-CrmA protein was detected using ECL detection reagents (Amersham).

Each sample of cells transfected with a CrmA expression plasmid showed high levels of Flag-CrmA expression, in contrast to parental wild type control cells (MG63-WT) which showed no expression.

### EXAMPLE 2

A PKR Overexpressing and a PKR and Anti-Apoptotic Protein Expressing Namalwa Cell Line

A. Preparation of pEF-FLAG-Bcl-X_{L}

The pEF-FLAG-Bcl-X_{L} vector (Huang, et al., 1997) contains a full length cDNA encoding the anti-apoptotic Bcl-X_{L} protein operably linked to the strong elongation factor 1 alpha (EF-1 alpha) promoter. An additional salient feature of the vector is the N-terminal FLAG epitope (Hopp et al., 1988) that was added to the Bcl-X_{L} protein to facilitate selection of cell lines that express high levels of Bcl-X_{L}.

The vector also includes i) a polyadenylation signal and transcription termination sequence to enhance mRNA stability; ii) a SV40 origin for episomal replication and simple vector rescue; iii) an ampicillin resistance gene and a ColE1 origin for selection and maintenance in E. coli; and iv) a puromycin resistance marker (Puro) to allow for selection and identification of the plasmid-containing eukaryotic cells after transfection of a Bcl-X_{L} and PKR.

B. Preparation of pcDNA-FLAG-PKR

The pcDNA-FLAG-PKR vector contains cDNA encoding the full-length human PKR molecule (551 amino acids; Meurs, et al., 1990; GenBank Accession No. NM002759) modified by the polymerase chain reaction to include the N terminal FLAG tag (Hopp et al., 1988) encoding the sequence MDYKDDDDK, and inserted into the eukaryotic expression vector pcDNA3 (Invitrogen), such that the FLAG-PKR coding sequence was expressed under the control of the CMV promoter.

The vector, termed pcDNA-FLAG-PKR, contains various features suitable for PKR transcription, including: i) a promoter sequence from the immediate early gene of the human CMV for high level mRNA expression; ii) a polyadenylation signal and transcription termination sequence from the bovine growth hormone (BGH) gene to enhance mRNA stability; iii) a SV40 origin for episomal replication and simple vector rescue; iv) an ampicillin resistance gene and a ColE1 origin for selection and maintenance in E. coli; and v) a G418 resistance marker (Neo) to allow for selection and identification of the plasmid-containing eukaryotic cells after transfection.

A second PKR vector, designated pTRE-PKR, was prepared by inserting the same PKR cDNA into a restriction/insertion site of a pTRE plasmid obtained from Clontech. The pTRE plasmid is similar to the pFLAG used in making the first-described PKR vector, but contains a tetracycline-responsive element upstream of the CMV promoter used to control the inserted gene. In the studies reported in Example 4, the TRE function was not exploited, and so the operation of the two PKR vectors in transformed cells was predicted to be essentially the same.

C. Preparation of the 6A Cell Line

The human B lymphoblastoid cell line Namalwa (WT) was transfected sequentially with the plasmids, pEF-FLAG-Bcl-X_{L} and pcDNA-FLAG-PKR. Stable transfectants were obtained by electroporation of 4×10⁶ exponentially growing Namalwa cells with 15 μg of the pEF-FLAG-Bcl-X_{L} plasmid in DMEM/F12 (+10% FBS) using a Gene Pulser apparatus (BioRad) set at 800 uF, 300V. Bulk populations of stable transformants were obtained by selection with 2 μg/ml puromycin (Gibco-BRL) for 3-4 weeks and screened for BCI-X_{L} expression by flow cytometry as follows. The bulk transfectants were washed, permeabilized with acetone and subsequently stained with 2 μg/ml mouse anti-FLAG M2 monoclonal antibody (IBI) and then with phycoerythrin conjugated goat anti-mouse IgG (1 μg/ml; Becton-Dickinson). Cells were analyzed in the FACScan, live and dead cells being discriminated on the basis of their forward and side light-scattering properties and Bcl-X_{L} expressing cells by their level of fluorescence intensity. High level Bcl-X_{L} expressing transformants (Namalwa-Bcl-X_{L}) were then transfected with pcDNA-FLAG-PKR.

Stable high level Bcl-X_{L} expressing transfectants were obtained by electroporation of 4×10⁶ exponentially growing Namalwa-Bcl-X_{L} cells with 15 μg of the pcDNA-FLAG-PKR plasmid in DMEM/F12 (+10% FBS) using a Gene Pulser apparatus (BioRad) set at 800PF, 300V. Bulk populations of stable transformants were obtained by selection with 2 mg/ml geneticin (G418, Gibco-BRL) for 3-4 weeks. Clonal lines were subsequently obtained by limiting dilution cloning and analyzed for Bcl-X_{L} and PKR expression by Western blot analysis (Huang et al., 1997). The proteins were identified using 2 μg/ml anti-FLAG M2 antibody followed by goat anti-mouse IgG-peroxidase conjugate and ECL detection (Amersham). An exemplary BCI-X_{L} and PKR positive cell line was designated 6A.

D. Preparation of the A9 Cell Line

Stable high level PKR expressing transfectants were obtained by electroporation of 4×10⁶ exponentially growing Namalwa cells with 15 μg of the pTRE-PKR plasmid in DMEM/F12 (+10% FBS) using a Gene Pulser apparatus (BioRad) set at 800 μF,300V. Bulk populations of stable transformants were obtained by selection with 2 mg/ml geneticin (G418, Gibco-BRL) for 3-4 weeks. Clonal lines were subsequently obtained by limiting dilution cloning and analyzed for PKR expression by Western blot analysis (Huang et al., 1997).

### EXAMPLE 3

Characterization of a Transgenic Bcl-X_{L}-and PKR-Expressing Namalwa Cell Line

1. Increased Cell Viability

Wildtype Namalwa cells (WT) and the A9 and 6A cells from Example 3 were examined for cell viability in culture under conditions of PKR overexpression and cytokine induction. Specifically, PKR and Bcl-X_{L} double-transfected Namalwa cells (the 6A cell line), PKR-transfected Namalwa cells (the A9 cell line) and parental Namalwa cells (WT) were cultured at 2.5×10⁵ cells/ml in DMEM/F12 medium supplemented with 10% FBS. The cells were treated with 20 mM PMA (priming agent) for 20 hr followed by treatment with either 200 μg/ml poly r(I):poly r(C) and 10 μg/ml DEAE Dextran (poly IC induction) for 72 hr or 200 HAU/1×10⁶ cells of Sendai virus for 48 hr. Following treatment, cell viability was assessed by flow cytometry on a FACScan.

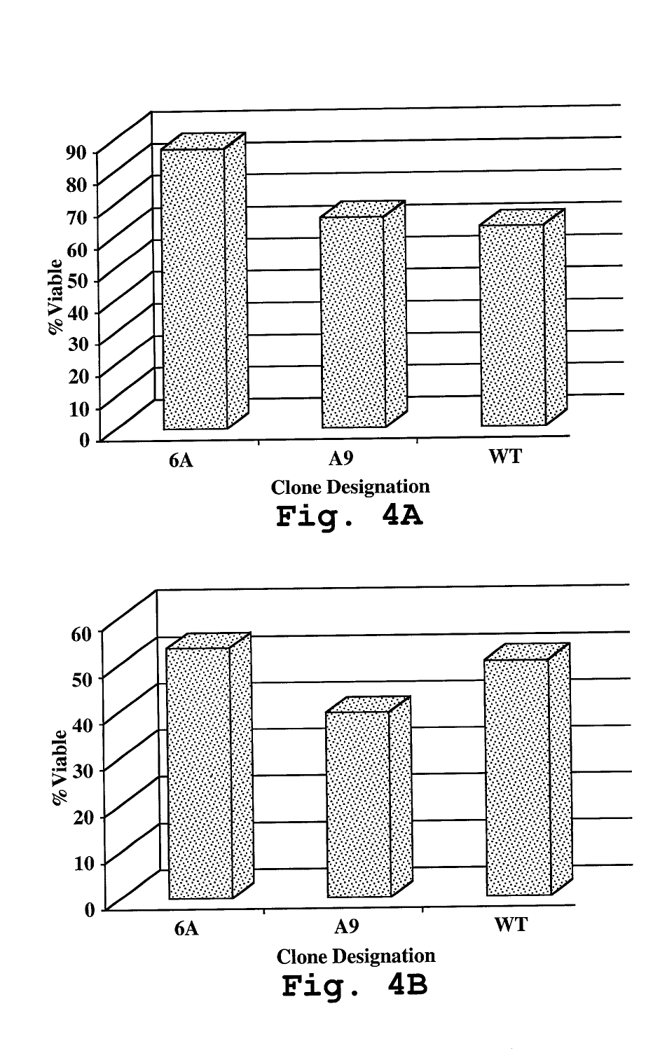

FIG. 4A shows that following Sendai virus induction, cell viability was similar for the PKR-transfected Namalwa cells (the A9 cell line) and parental Namalwa cells (WT), with greater viability observed for the PKR and Bcl-X_{L} double-transfected Namalwa cells (the 6A cell line).

FIG. 4B shows that following poly IC induction, cell viability was similar for the PKR and Bcl-X_{L} double-transfected Namalwa cells (the 6A cell line) and parental Namalwa cells (WT), with lower viability observed for PKR-transfected Namalwa cells (the A9 cell line).

2. Increased Expression of Interferon-alpha

The level of IFN-alpha production was also analyzed in the three cell lines following cytokine induction by poly IC and Sendai virus, both under conditions of PKR overproduction. The culture supernatants were collected and analyzed for IFN-alpha levels by ELISA according to the procedure provided by the supplier of the ELISA kits (R&D Systems).

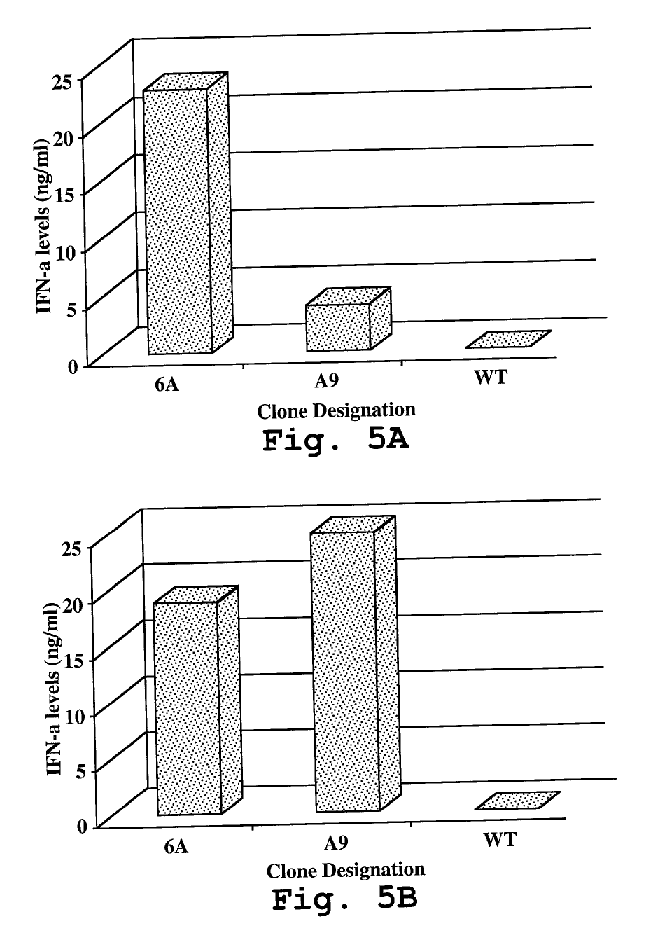

The results shown in FIG. 5A indicate that following Sendai virus induction, IFN-alpha production by PKR and Bcl-X_{L} double-transfected Namalwa cells (the 6A cell line) was significantly greater than IFN-alpha production by PKR-transfected Namalwa cells (the A9 cell line) and parental Namalwa cells (WT).

The results shown in FIG. 5B indicate that following poly IC induction, IFN-alpha production by PKR-transfected Namalwa cells (the A9 cell line) and PKR and Bcl-X_{L} double-transfected Namalwa cells (the 6A cell line) was significantly greater than IFN-alpha production by parental Namalwa cells (WT).

From the foregoing, it can be seen how various objects and features of the invention are met. Those skilled in the art can now appreciate from the foregoing description that the broad teachings of the present invention can be implemented in a variety of forms. Therefore, while this invention has been described in connection with particular embodiments and examples thereof, the true scope of the invention should not be so limited. Various changes and modification may be made without departing from the scope of the invention, as defined by the appended claims.

It is claimed:

1. A human cell composition for use in producing one or more cytokines, comprising:
a human cell line characterized by expression of the coding sequence for an anti-apoptotic protein and a level of cytokine production that is at least two times (2×) the level of cytokine production exhibited by a corresponding parental cell line that does not express the coding sequence for the anti-apoptotic protein.

2. The cell line composition according to claim 1, wherein said anti-apoptotic protein is CrmA.

3. A human cell composition for use in producing one or more cytokines, prepared by the process of:
(a) obtaining a parental human cell line capable of producing one or more cytokines;
(b) modifying the cells by introducing a first expression vector comprising the (i) coding sequence for CrmA operably linked to a first promoter, (ii) a first selectable marker-encoding nucleic acid sequence, and (iii) additional control elements necessary for expression in human cells, into the cells of said cell line; and
(c) culturing said modified cells in medium containing a first selection agent to select for CrmA-expressing cells.

4. A human cell composition prepared by the process of claim 3, further comprising the step of:
(d) treating said CrmA-expressing cells in a manner effective to result in enhanced cytokine production, wherein said transformed and treated cell line is characterized by a level of cytokine production that is at least two times (2×) the level of cytokine production by the corresponding non-transformed parental cell line.

5. A human cell composition prepared by the process of claim 3, further comprising the step of:
(d) further modifying said CrmA-expressing cell line by introducing a second expression vector comprising (i) the coding sequence for PKR operably linked to a second promoter; (ii) a second selectable marker-encoding nucleic acid sequence; and (iii) additional control elements necessary for expression in human cells into the cells of said cell line; and
(e) culturing said further modified cells in medium containing a selection agent specific for said second selectable marker to select for PKR overexpressing cells.

6. A human cell composition prepared by the process of claim 5, further comprising the step of:
(f) treating said PKR overexpressing cells in a manner effective to result in enhanced cytokine production, wherein said transformed and treated cell line is characterized by a level of cytokine production that is at least two times (2×) the level of cytokine production by the corresponding non-transformed parental cell line.

7. A human cell composition prepared by the process of claim 4 or 6, wherein treating means subjecting said transformed cells to one or both of priming and inducing.

8. The human cell composition according to claim 7, wherein priming means exposing said transformed cells to phorbol myristate acetate (PMA) or interferon-β.

9. The human cell composition according to claim 7, wherein inducing means exposing said transformed cells to a microbial inducing agent selected from the group consisting of Sendai virus, encephalomyocarditis virus and Herpes simplex virus.

10. The human cell composition according to claim 9, wherein said microbial inducing agent is Sendai virus.

11. The human cell composition according to claim 7, wherein inducing means exposing said cells to at least one non-microbial inducing agent selected from the group consisting of poly(I):poly(C) (poly IC), or poly r(I):poly r(C) (poly rIC), heparin, dextran sulfate, cycloheximide, Actinomycin D, sodium butyrate, a calcium ionophore and chondroitin sulfate.

12. The human cell composition according to claim 11, wherein inducing means exposing said cells to polyI:C, cycloheximide and Actinomycin D.

13. In an improved method for producing one or more cytokines in human cell culture, the improvement directed to increasing cell viability and the amount of cytokine production, by culturing a parental human cell under conditions of one or more of (i) modification effective to result in anti-apoptotic protein expression; (ii) modification effective to result in cytokine regulatory factor overexpression; (iii) priming; and (iv) inducing, wherein the amount of cytokine production is at least two times (2×) the level of cytokine production by the corresponding non-transformed parental cell line.

14. The method according to claim 13, wherein modification effective to result in anti-apoptotic protein expression means introducing a first expression vector comprising the (i) coding sequence for CrmA operably linked to a first promoter, (ii) a first selectable marker-encoding nucleic acid sequence, and (iii) additional control elements necessary for expression of CrmA in human cells into the cells of said cell line, and culturing the cells in medium containing a first selection agent to select for CrmA-expressing cells.

15. The method according to claim 14, wherein modification effective to result in cytokine regulatory factor overexpression means introducing a second expression vector comprising (i) the coding sequence for PKR operably linked to a second promoter; (ii) a second selectable marker-encoding nucleic acid sequence; and (iii) additional control elements necessary for expression in human cells into cells of said CrmA-expressing cell line, and culturing the cells in medium containing a selection agent specific for said second selectable marker to select for PKR-overexpressing cells.

16. The method according to claim 14 or 15, further comprising priming the cells by exposing them to one or both of phorbol myristate acetate (PMA) and interferon-β.

17. The method according to claim 15 or 16, further comprising inducing the cells by exposing them to a microbial inducing agent selected from the group consisting of Sendai virus, encephalomyocarditis virus and Herpes simplex virus.

18. The method according to any one of claims 16 or 17, further comprising inducing the cells by exposing them to at least one non-microbial inducing agent selected from the group consisting of poly(I):poly(C) (poly IC), or poly r(I):poly r(C) (poly rIC), heparin, dextran sulfate, cycloheximide, Actinomycin D, sodium butyrate, a calcium ionophore and chondroitin sulfate.

19. The method of according to claim any one of claims 17 or 18, wherein the one or more cytokine(s) are selected from the group consisting of interferon-alpha (IFN-alpha), interferon-beta (IFN-beta), interferon-gamma (IFN-gamma); granulocyte macrophage colony stimulating factor (GM-CSF); granulocyte colony stimulating factor (G-CSF); interleukin-2 (IL2); interleukin-3 (IL-3); interleukin-7 (IL-7); interleukin-8 (IL-8); interleukin-10 (IL-10); and interleukin-12 (IL-12).
